(19) United States
(12) Patent Application Publication
(10) Pub. No.: US 2013/0303369 A1
(43) Pub. Date: Nov. 14, 2013

(54) Fatty Amine Salts Of Herbicidal Pyrimidines

(71) Applicant: E. I. du Pont de Nemours and Company, (US)

(72) Inventor: Carlos F. Barrientos, Dublin, OH (US)

(73) Assignee: E. I. du Pont de Nemours and Company, Wilmington, DE (US)

(21) Appl. No.: 13/728,603

(22) Filed: Dec. 27, 2012

Related U.S. Application Data
(60) Provisional application No. 61/581,154, filed on Dec. 29, 2011.

(51) Int. Cl. A01N 43/54; C07D 239/42

(52) U.S. Cl. CPC A01N 43/54; C07D 239/42
USPC 504/103; 544/329; 504/136; 504/239; 504/134

(57) ABSTRACT

This invention is directed to (1) a soluble intermediate prepared from reaction of the acid form of one or more of the compounds disclosed by Formula I given below with at least one long-chain fatty amine; (2) a process for solubilizing one or more of the compounds disclosed by Formula I given below by reacting said compounds with at least one long-chain fatty amine to form a fatty-amine salt of Formula I compound; and (3) a finished pesticide product comprising at least one of the soluble intermediates, that is, fatty-amine salt of Formula I compound.

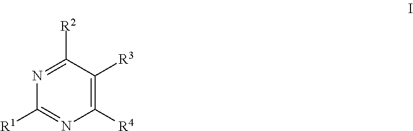

wherein
R¹ is cyclopropyl optionally substituted with 1-5 R⁵, isopropyl optionally substituted with 1-5 R⁶, or phenyl optionally substituted with 1-3 R⁷;
R² is ((O)ⱼC(R¹⁵)(R¹⁶))ₖR;
R is CO₂H or a herbicidally effective derivative of CO₂H;
R³ is halogen, cyano, nitro, OR²⁰, SR²¹ or N(R²²)R²³;
R⁴ is —N(R²⁴)R²⁵ or —NO₂;
j is 0 or 1; and k is 0 or 1; provided that when k is 0, then j is 0;
and R⁵, R⁶, R⁷, R¹⁵, R¹⁶, R²⁰, R²¹, R²², R²³, R²⁴ and R²⁵ are as defined in the disclosure.

### FIELD OF THE INVENTION

The present invention relates to a process for preparing agriculturally suitable salts and compositions from certain pyrimidines, an improved finished pesticide product made from such salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, corn (maize), potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds which are more effective, less costly, less toxic, environmentally safer or have different modes of action.

World Patent Application Publication WO 92/05159-A discloses pyrimidines useful as plant protectants, especially fungicides. European Patent Application Publication EP-136976-A2 discloses pyrimidines as plant growth regulators. U.S. Pat. No. 5,324,710 discloses sulfonated heterocyclic carboxamide derivatives of pyrimidines as herbicides and growth regulators.

### SUMMARY OF THE INVENTION

This invention is directed to (1) a soluble intermediate prepared from reaction of the acid form of one or more of the compounds disclosed by Formula I given below with at least one long-chain fatty amine; (2) a process for solubilizing one or more of the compounds disclosed by Formula I given below by reacting said compounds with at least one long-chain fatty amine to form a fatty-amine salt of Formula I compound; and (3) a finished pesticide product comprising at least one of the soluble intermediates, that is, fatty-amine salt of Formula I compound.

Formula I compounds are further defined in the “Detailed Description of Invention” section, infra. It is to be understood that Formula I, representing a compound also represents said compound's all geometric isomers, stereoisomers, and racemic mixtures, if any.

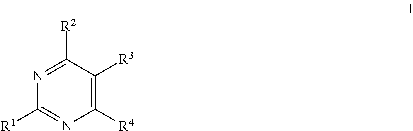

wherein
R¹ is cyclopropyl optionally substituted with 1-5 R⁵, isopropyl optionally substituted with 1-5 R⁶, or phenyl optionally substituted with 1-3 R⁷;
R² is ((O)ⱼC(R¹⁵)(R¹⁶))ₖR;
R is CO₂H or a herbicidally effective derivative of CO₂H;
R³ is halogen, cyano, nitro, OR²⁰, SR²¹ or N(R²²)R²³;
R⁴ is —N(R²⁴)R²⁵ or —NO₂;
each R⁵ and R⁶ is independently halogen, C₁-C₆ alkyl, C₁-C₆ haloalkyl, C₂-C₆ alkenyl, C₂-C₆ haloalkenyl, C₁-C₃ alkoxy, C₁-C₂ haloalkoxy, C₁-C₃ alkylthio or C₁-C₂ haloalkylthio;
each R⁷ is independently halogen, cyano, nitro, C₁-C₄ alkyl, C₁-C₄ haloalkyl, C₃-C₆ cycloalkyl, C₃-C₆ halocycloalkyl, C₁-C₄ hydroxyalkyl, C₂-C₄ alkoxyalkyl, C₂-C₄ haloalkoxyalkyl, C₂-C₄ alkenyl, C₂-C₄ haloalkenyl, C₃-C₄ alkynyl, C₃-C₄ haloalkynyl, hydroxy, C₁-C₄ alkoxy, C₁-C₄ haloalkoxy, C₂-C₄ alkenyloxy, C₂-C₄ haloalkenyloxy, C₃-C₄ alkynyloxy, C₃-C₄ haloalkynyloxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂-C₄ alkenylthio, C₂-C₄ haloalkenylthio, C₂-C₄ alkenylsulfinyl, C₂-C₄ haloalkenylsulfinyl, C₂-C₄ alkenylsulfonyl, C₂-C₄ haloalkenylsulfonyl, C₃-C₄ alkynylthio, C₃-C₄ haloalkynylthio, C₃-C₄ alkynylsulfinyl, C₃-C₄ haloalkynylsulfinyl, C₃-C₄ alkynylsulfonyl, C₃-C₄ haloalkynylsulfonyl, C₁-C₄ alkylamino, C₂-C₈ dialkylamino, C₃-C₆ cycloalkylamino, C₄-C₆ (alkyl)cycloalkylamino, C₂-C₆ alkylcarbonyl, C₂-C₆ alkoxycarbonyl, C₂-C₆ alkylaminocarbonyl, C₃-C₈ dialkylaminocarbonyl, C₃-C₆ trialkylsilyl, phenyl, phenoxy and 5- or 6-membered heteroaromatic rings, each phenyl, phenoxy and 5- or 6-membered heteroaromatic ring optionally substituted with one to three substituents independently selected from R⁴⁵; or
two adjacent R⁷ are taken together as —OCH₂O—, —CH₂CH₂O—, —OCH(CH₃)O—, —OC(CH₃)₂O—, —OCF₂O—, —CF₂CF₂O—, —OCF₂CF₂O— or —CH═CH—CH═CH—;
R¹⁵ is H, halogen, C₁-C₄ alkyl, C₁-C₄ haloalkyl, hydroxy, C₁-C₄ alkoxy or C₂-C₄ alkylcarbonyloxy;
R¹⁶ is H, halogen, C₁-C₄ alkyl or C₁-C₄ haloalkyl; or
R¹⁵ and R¹⁶ are taken together as an oxygen atom to form, with the carbon atom to which they are attached, a carbonyl moiety;
R²⁰ is H, C₁-C₄ alkyl or C₁-C₃ haloalkyl;
R²¹ is H, C₁-C₄ alkyl or C₁-C₃ haloalkyl;
R²² and R²³ are independently H or C₁-C₄ alkyl;
R²⁴ is H, C₁-C₄ alkyl optionally substituted with 1-2 R³⁰, C₂-C₄ alkenyl optionally substituted with 1-2 R³¹, or C₂-C₄ alkynyl optionally substituted with 1-2 R³²; or R²⁴ is C(═O)R³³, nitro, OR³⁴, S(O)₂R³⁵, N(R³⁶)R³⁷ or N═C(R⁶²)R⁶³;
R²⁵ is H, C₁-C₄ alkyl optionally substituted with 1-2 R³⁰ or C(═O)R³³; or
R²⁴ and R²⁵ are taken together as a radical selected from —(CH₂)₄—, —(CH₂)₅—, —CH₂CH═CHCH₂— and —(CH₂)₂O(CH₂)₂—, each radical optionally substituted with 1-2 R³⁸; or
R²⁴ and R²⁵ are taken together as ═C(R³⁹)N(R⁴⁰)R⁴¹ or ═C(R⁴²)OR⁴³;
each R³⁰, R³¹ and R³² is independently halogen, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
each R³³ is independently H, C₁-C₁₄ alkyl, C₁-C₃ haloalkyl, C₁-C₄ alkoxy, phenyl, phenoxy or benzyloxy;
R³⁴ is H, C₁-C₄ alkyl, C₁-C₃ haloalkyl or CHR⁶⁶C(O)OR⁶⁷;
R³⁵ is C₁-C₄ alkyl or C₁-C₃ haloalkyl;
R³⁶ is H, C₁-C₄ alkyl or C(═O)R⁶⁴;
R³⁷ is H or C₁-C₄ alkyl;
each R³⁸ is independently halogen, C₁-C₃ alkyl, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
R³⁹ is H or C₁-C₄ alkyl;
R⁴⁰ and R⁴¹ are independently H or C₁-C₄ alkyl; or
R⁴⁰ and R⁴¹ are taken together as —(CH₂)₄—, —(CH₂)₅—, —CH₂CH═CHCH₂— or —(CH₂)₂O(CH₂)₂—;
R⁴² is H or C₁-C₄ alkyl;
R⁴³ is C₁-C₄ alkyl;
each R⁴⁵ is independently halogen, cyano, nitro, C₁-C₄ alkyl, C₁-C₄ haloalkyl, C₃-C₆ cycloalkyl, C₃-C₆ halocycloalkyl, C₂₋₄ alkenyl, C₂-C₄ haloalkenyl, C₃-C₄ alkynyl, C₃-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁶² is H, C₁-C₄ alkyl or phenyl optionally substituted with 1-3 R⁶⁵;
R⁶³ is H or C₁-C₄ alkyl; or
R⁶² and R⁶³ are taken together as —(CH₂)₄— or —(CH₂)₅—;
R⁶⁴ is H, C₁-C₁₄ alkyl, C₁-C₃ haloalkyl, C₁-C₄ alkoxy, phenyl, phenoxy or benzyloxy;
each R⁶⁵ is independently CH₃, Cl or OCH₃;
R⁶⁶ is H, C₁-C₄ alkyl or C₁-C₄ alkoxy;
R⁶⁷ is H, C₁-C₄ alkyl or benzyl;
j is 0 or 1; and
k is 0 or 1;

provided that:
(a) when k is 0, then j is 0;
(b) when R² is CH₂OR^{a} wherein R^{a} is H, optionally substituted alkyl or benzyl, then R³ is other than cyano;
(c) when R¹ is phenyl substituted by Cl in each of the meta positions, the phenyl is also substituted by R⁷ in the para position;
(d) when R¹ is phenyl substituted by R⁷ in the para position, said R⁷ is other than tert-butyl, cyano or optionally substituted phenyl;
(e) when R¹ is cyclopropyl or isopropyl optionally substituted with 1-5 R⁶, then R is other than C(═W)N(R^{b})S(O)₂—R^{c}-R^{d} wherein W is O, S, NR^{e} or NORₑ; R^{b} is hydrogen, C₁-C₄ alkyl, C₂-C₆ alkenyl or C₂-C₆ alkynyl; R^{c} is a direct bond or CHR^{f}, O, NR^{e} or NOR^{e}; R^{d} is an optionally substituted heterocyclic or carbocyclic aromatic radical having 5 to 6 ring atoms, the radical being optionally condensed with an aromatic or nonaromatic 5- or 6-membered ring; each R^{e} is independently H, C₁-C₃ alkyl, C₁-C₃ haloalkyl or phenyl; and R^{f} is H, C₁-C₃ alkyl or phenyl; and
(f) the compound of Formula I is other than diethyl 6-amino-5-nitro-2-phenyl-4-pyrimidinemalonate.

In one embodiment of the invention, the process for making said fatty-amine salt from Formula I compound comprises reaction of said at least one Formula I compound with said at least one long-chain fatty amine under high-shear conditions.

This invention also relates to a herbicidal composition comprising a herbicidally effective amount of a fatty-amine salt of at least one Formula I compound and at least one of a surfactant, a solid diluent or a liquid diluent. This invention further relates to a method for controlling the growth of undesired vegetation comprising contacting the vegetation or its environment with a herbicidally effective amount of a fatty-amine salt of at least one Formula I compound (e.g., as a composition described herein). This invention also relates to a herbicidal mixture comprising a herbicidally effective amount of a fatty-amine salt of at least one Formula I compound and an effective amount of at least one additional active ingredient selected from the group consisting of another herbicide and a herbicide safener. This invention further relates to a herbicidal composition comprising a herbicidally effective amount of a fatty-amine salt of at least one Formula I compound, an effective amount of at least one additional active ingredient selected from the group consisting of another herbicide and a herbicide safener, and at least one of a surfactant, a solid diluent or a liquid diluent.

### DETAILS OF THE INVENTION

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Formula I compound is further described below:

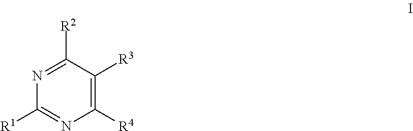

wherein
R¹ is cyclopropyl optionally substituted with 1-5 R⁵, isopropyl optionally substituted with 1-5 R⁶, or phenyl optionally substituted with 1-3 R⁷;
R² is ((O)C(R¹⁵)(R¹⁶))ₖR;
R is CO₂H or a herbicidally effective derivative of CO₂H;
R³ is halogen, cyano, nitro, OR²⁰, SR²¹ or N(R²²)R²³;
R⁴ is —N(R²⁴)R²⁵ or —NO₂;
each R⁵ and R⁶ is independently halogen, C₁-C₆ alkyl, C₁-C₆ haloalkyl, C₂-C₆ alkenyl, C₂-C₆ haloalkenyl, C₁-C₃ alkoxy, C₄-C₂ haloalkoxy, C₁-C₃ alkylthio or C₁-C₂ haloalkylthio;
each R⁷ is independently halogen, cyano, nitro, C₁-C₄ alkyl, C₁-C₄ haloalkyl, C₃-C₆ cycloalkyl, C₃-C₆ halocycloalkyl, C₁-C₄ hydroxyalkyl, C₂-C₄ alkoxyalkyl, C₂-C₄ haloalkoxyalkyl, C₂-C₄ alkenyl, C₂-C₄ haloalkenyl, C₃-C₄ alkynyl, C₃-C₄ haloalkynyl, hydroxy, C₁-C₄ alkoxy, C₁-C₄ haloalkoxy, C₂-C₄ alkenyloxy, C₂-C₄ haloalkenyloxy, C₃-C₄ alkynyloxy, C₃-C₄ haloalkynyloxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂-C₄ alkenylthio, C₂-C₄ haloalkenylthio, C₂-C₄ alkenylsulfinyl, C₂-C₄ haloalkenylsulfinyl, C₂-C₄ alkenylsulfonyl, C₂-C₄ haloalkenylsulfonyl, C₃-C₄ alkynylthio, C₃-C₄ haloalkynylthio, C₃-C₄ alkynylsulfinyl, C₃-C₄ haloalkynylsulfinyl, C₃-C₄ alkynylsulfonyl, C₃-C₄ haloalkynylsulfonyl, C₁-C₄ alkylamino, C₂-C₈ dialkylamino, C₃-C₆ cycloalkylamino, C₄-C₆ (alkyl)cycloalkylamino, C₂-C₆ alkylcarbonyl, C₂-C₆ alkoxycarbonyl, C₂-C₆ alkylaminocarbonyl, C₃-C₈ dialkylaminocarbonyl, C₃-C₆ trialkylsilyl, phenyl, phenoxy and 5- or 6-membered heteroaromatic rings, each phenyl, phenoxy and 5- or 6-membered heteroaromatic ring optionally substituted with one to three substituents independently selected from R⁴⁵; or
two adjacent R⁷ are taken together as —OCH₂O—, —CH₂CH₂O—, —OCH(CH₃)O—, —OC(CH₃)₂O—, —OCF₂O—, —CF₂CF₂O—, —OCF₂CF₂O— or —CHCH—CH═CH—;
R¹⁵ is H, halogen, C₁-C₄ alkyl, C₁-C₄ haloalkyl, hydroxy, C₁-C₄ alkoxy or C₂-C₄ alkylcarbonyloxy;
R¹⁶ is H, halogen, C₁-C₄ alkyl or C₁-C₄ haloalkyl; or
R¹⁵ and R¹⁶ are taken together as an oxygen atom to form, with the carbon atom to which they are attached, a carbonyl moiety;
R²⁰ is H, C₁-C₄ alkyl or C₁-C₃ haloalkyl;
R²¹ is H, C₁-C₄ alkyl or C₁-C₃ haloalkyl;
R²² and R²³ are independently H or C₁-C₄ alkyl;
R²⁴ is H, C₁-C₄ alkyl optionally substituted with 1-2 R³⁰, C₂-C₄ alkenyl optionally substituted with 1-2 R³¹, or C₂-C₄ alkynyl optionally substituted with 1-2 R³²; or R²⁴ is C(═O)R³³, nitro, OR³⁴, S(O)₂R³⁵, N(R³⁶)R³⁷ or N═C(R⁶²)R⁶³
R²⁵ is H, C₁-C₄ alkyl optionally substituted with 1-2 R³⁰ or C(═O)R³³; or
R²⁴ and R²⁵ are taken together as a radical selected from —(CH₂)₄—, —(CH₂)₅—, —CH₂CH═CHCH₂— and —(CH₂)₂O(CH₂)₂—, each radical optionally substituted with 1-2 R³⁸; or
R²⁴ and R²⁵ are taken together as ═C(R³⁹)N(R⁴⁰)R⁴¹ or ═C(R⁴²)OR⁴³;
each R³⁰, R³¹ and R³² is independently halogen, C₁-C₃ alkoxy, C₁₋₃haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
each R³³ is independently H, C₁-C₁₄ alkyl, C₁-C₃ haloalkyl, C₁-C₄ alkoxy, phenyl, phenoxy or benzyloxy;
R³⁴ is H, C₁-C₄ alkyl, C₁-C₃ haloalkyl or CHR⁶⁶C(O)OR⁶⁷;
R³⁵ is C₁-C₄ alkyl or C₁-C₃ haloalkyl;
R³⁶ is H, C₁-C₄ alkyl or C(═O)R⁶⁴;
R³⁷ is H or C₁-C₄ alkyl;
each R³⁸ is independently halogen, C₁-C₃ alkyl, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
R³⁹ is H or C₁-C₄ alkyl;
R⁴⁰ and R⁴¹ are independently H or C₁-C₄ alkyl; or
R⁴⁰ and R⁴¹ are taken together as —(CH₂)₄—, —(CH₂)₅—, —CH₂CH═CHCH₂— or —(CH₂)₂O(CH₂)₂—;
R⁴² is H or C₁-C₄ alkyl;
R⁴³ is C₁-C₄ alkyl;
each R⁴⁵ is independently halogen, cyano, nitro, C₁-C₄ alkyl, C₁-C₄ haloalkyl, C₃-C₆ cycloalkyl, C₃-C₆ halocycloalkyl, C₂-C₄ alkenyl, C₂-C₄ haloalkenyl, C₃-C₄ alkynyl, C₃-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁶² is H, C₁-C₄ alkyl or phenyl optionally substituted with 1-3 R⁶⁵;
R⁶³ is H or C₁-C₄ alkyl; or
R⁶² and R⁶³ are taken together as —(CH₂)₄— or —(CH₂)₅—;
R⁶⁴ is H, C₁-C₁₄ alkyl, C₁-C₃ haloalkyl, C₁-C₄ alkoxy, phenyl, phenoxy or benzyloxy;
each R⁶⁵ is independently CH₃, Cl or OCH₃;
R⁶⁶ is H, C₁-C₄ alkyl or C₁-C₄ alkoxy;
R⁶⁷ is H, C₁-C₄ alkyl or benzyl;
j is 0 or 1; and
k is 0 or I;

provided that:
(a) when k is 0, then j is 0;
(b) when R² is CH₂OR^{a} wherein R^{a} is H, optionally substituted alkyl or benzyl, then R³ is other than cyano;
(c) when R¹ is phenyl substituted by Cl in each of the meta positions, the phenyl is also substituted by R⁷ in the para position;
(d) when R¹ is phenyl substituted by R⁷ in the para position, said R⁷ is other than tert-butyl, cyano or optionally substituted phenyl;
(e) when R¹ is cyclopropyl or isopropyl optionally substituted with 1-5 R⁶, then R is other than C(═W)N(R^{b})S(O)₂—R^{c}-R^{d} wherein W is O, S, NR^{e} or NOR^{e}; R^{b} is hydrogen, C₁-C₄ alkyl, C₂-C₆ alkenyl or C₂-C₆ alkynyl; R^{c} is a direct bond or CHR^{f}, O, NR^{e} or NOR^{e}; R^{d} is an optionally substituted heterocyclic or carbocyclic aromatic radical having 5 to 6 ring atoms, the radical being optionally condensed with an aromatic or nonaromatic 5- or 6-membered ring; each R^{e} is independently H, C₁-C₃ alkyl, C₁-C₃ haloalkyl or phenyl; and R^{f} is H, C₁-C₃ alkyl or phenyl; and
(f) the compound of Formula I is other than diethyl 6-amino-5-nitro-2-phenyl-4-pyrimidinemalonate.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂ and CH₃CH₂OCH₂CH₂. “Alkenyloxy” includes straight-chain or branched alkenyloxy moieties. Examples of “alkenyloxy” include H₂C═CHCH₂O, (CH₃)CH═CHCH₂O and CH₂═CHCH₂CH₂O. “Alkynyloxy” includes straight-chain or branched alkynyloxy moieties. Examples of “alkynyloxy” include HC≡CCH₂O and CH₃C≡CCH₂O. “Alkylthio” includes branched or straight-chain alkylthio moieties such as methylthio, ethylthio, and the different propylthio and butylthio isomers. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl isomers. “Alkylamino”, “dialkylamino”, “alkenylthio”, “alkenylsulfinyl”, “alkenylsulfonyl”, “alkynylthio”, “alkynylsulfinyl”, “alkynylsulfonyl”, and the like, are defined analogously to the above examples. “Cycloalkyl” includes, for example, cyclopropyl, cyclobutyl, cyclopentyl, and cyclohexyl. Examples of “cycloalkylalkyl” include cyclopropylmethyl, cyclopentylethyl, and other cycloalkyl moieties bonded to straight-chain or branched alkyl groups. “Alkylcycloalkyl” denotes alkyl substitution on a cycloalkyl moiety. Examples include 4-methylcyclohexyl and 3-ethylcyclopentyl. The term “heteroaromatic ring” includes fully aromatic heterocycles. Aromatic indicates that each of the ring atoms is essentially in the same plane and has a p-orbital perpendicular to the ring plane, and in which (4n+2) it electrons, when n is 0 or a positive integer, are associated with the ring to comply with Hickel's rule. The term carbocyclic aromatic radical is synonymous with the term isocyclic aromatic radical. A wide variety of synthetic methods are known in the art to enable preparation of aromatic heterocyclic rings; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996. The 5- and 6-membered heteroaromatic rings described for R⁷ typically comprise 1 to 4 heteroatom ring members, the heteroatom members selected from 0-4 N, 0-1 O and 0-1 S atoms. Exhibit 1 shows examples of heteroaromatic rings; H-1 through H-55 are to be construed as illustrative rather than limiting of the heteroaromatic rings within the scope of the present invention.

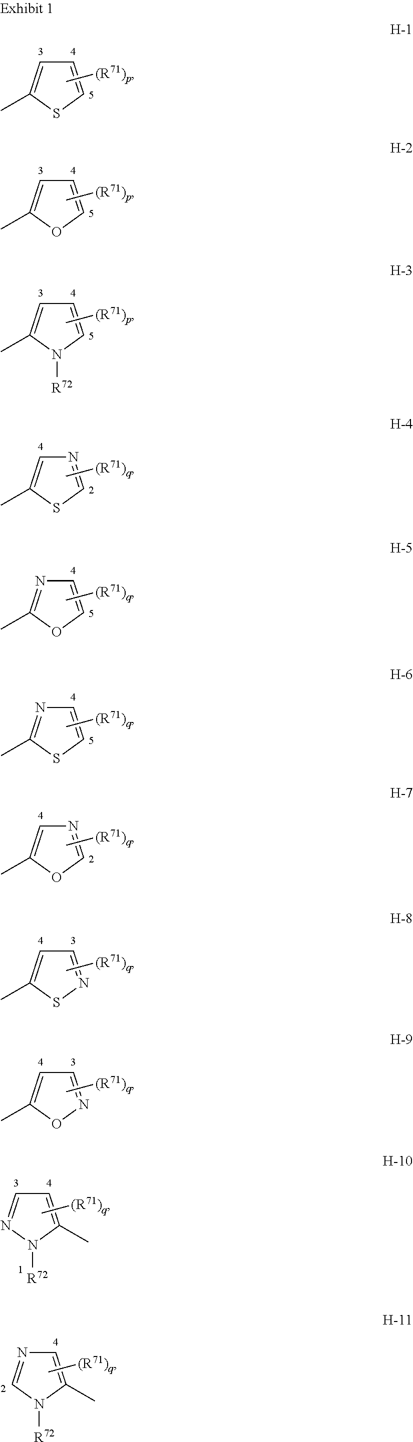

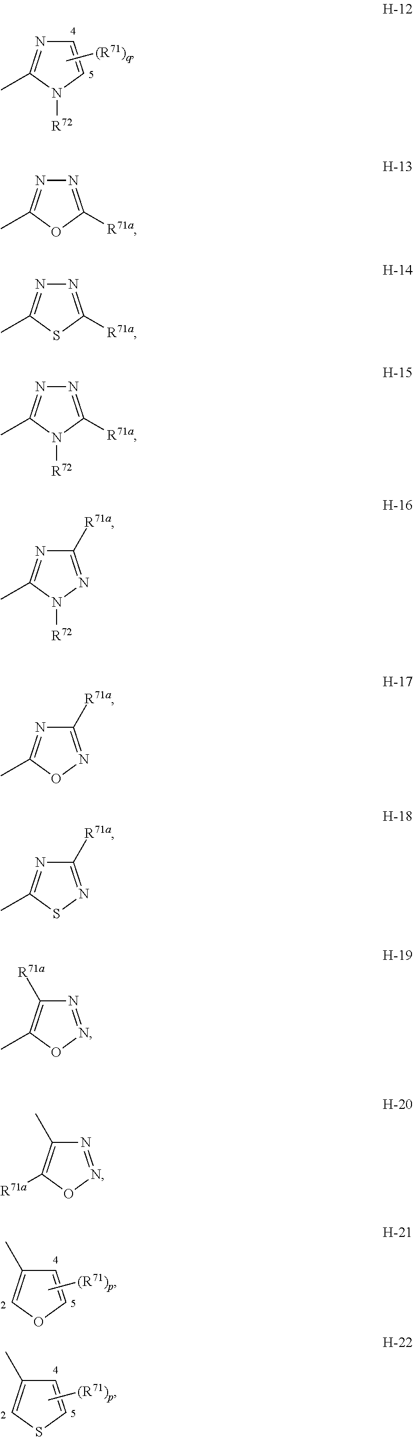

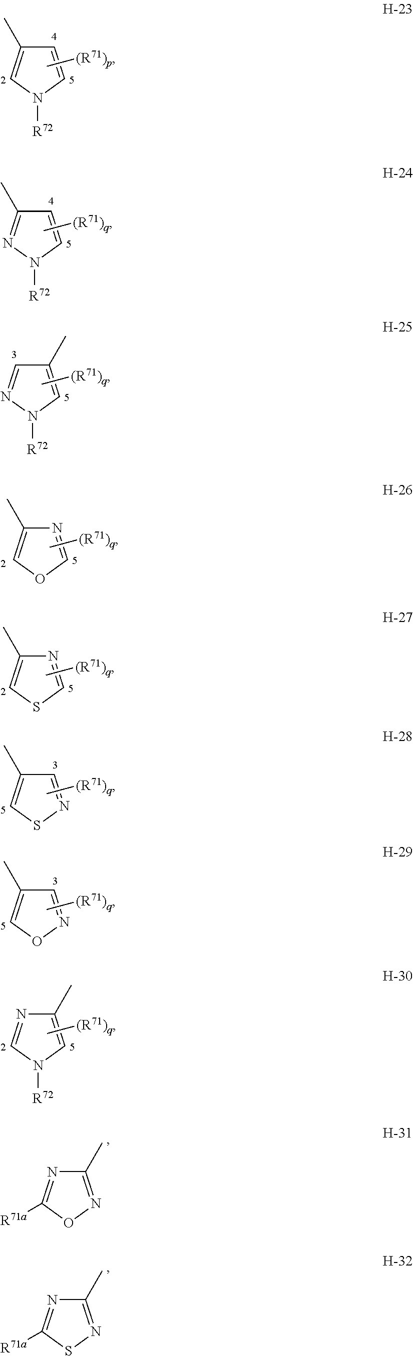

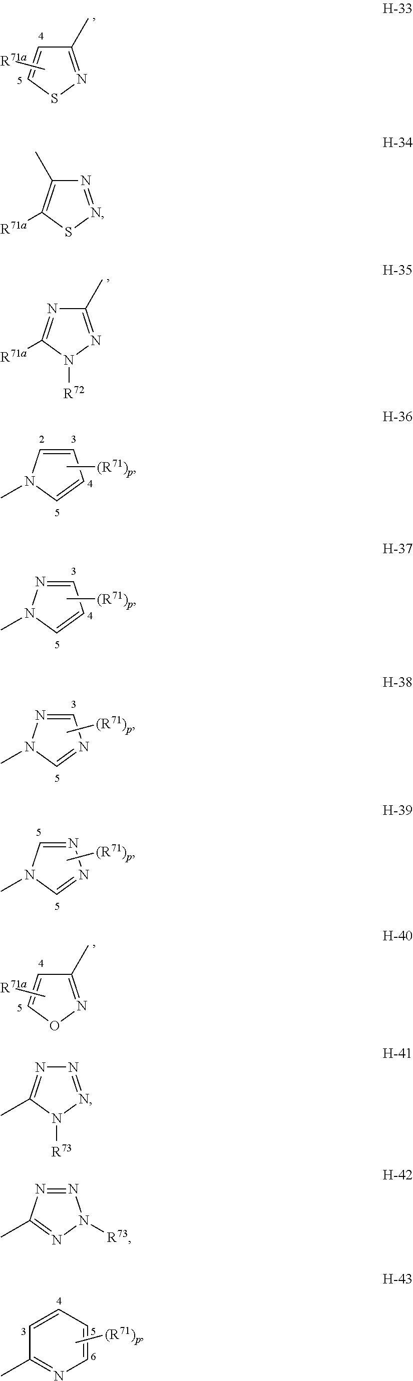

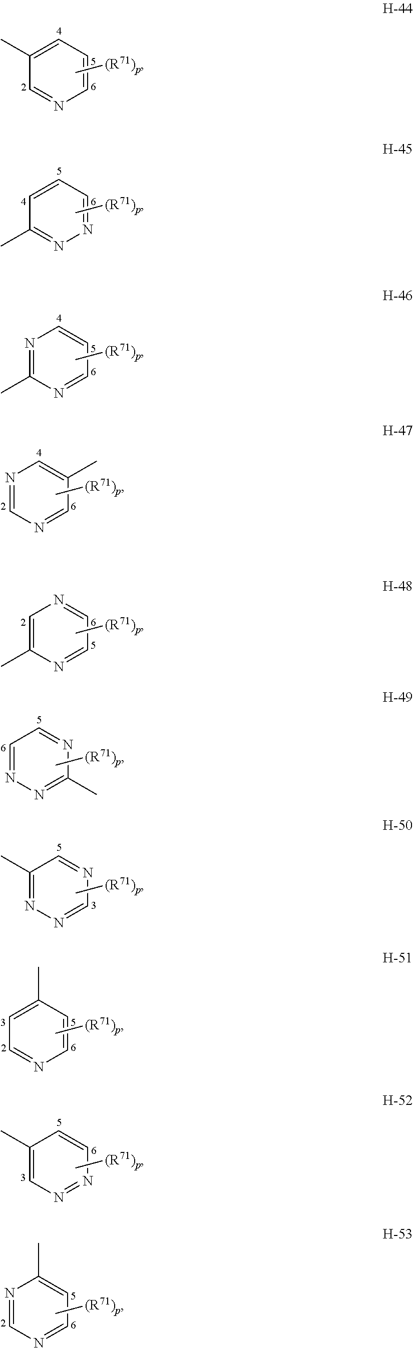

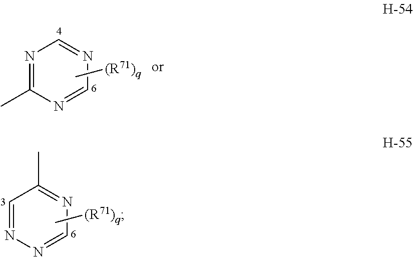

wherein

each R⁷¹ is independently R⁴⁵;

R^{71a}, R⁷² and R⁷³ are independently H or R⁴⁵;

p is an integer from 0 to 3; and

q is an integer from 0 to 2.

References herein to R⁷ groups H-1 through H-55 refer to those shown in Exhibit 1.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, “haloalkylthio”, and the like, are defined analogously to the term “haloalkyl”. Examples of “haloalkenyl” include (Cl)₂CCHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O₎ and CF₃CF₂S(O). Examples of “haloalkylsulfonyl” include CF₃S(O)₂, CCl₃S(O)₂, CF₃CH₂S(O)₂ and CF₃CF₂S(O)₂.

The total number of carbon atoms in a substituent group is indicated by the “Cᵢ-Cⱼ” prefix where i and j are numbers from 1 to 14. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. Examples of “alkylcarbonyl” include C(O)CH₃, C(O)CH₂CH₂CH₃ and C(O)CH(CH₃)₂. Examples of “alkoxycarbonyl” include CH₃C(═O), CH₃CH₂OC(═O), CH₃CH₂CH₂C(═O), (CH₃)₂CHOC(═O) and the different butoxy- or pentoxycarbonyl isomers. In the above recitations, when a compound of Formula I is comprised of one or more heterocyclic rings, all substituents are attached to these rings through any available carbon or nitrogen by replacement of a hydrogen on said carbon or nitrogen.

When a compound is substituted with a substituent bearing a subscript (e.g., (R^{d})₁₋₃) that indicates the number of instances (i.e. occurrences) of said substituent can vary or the substituent is preceded with a numeric range (e.g., 1-3 R^{d}) indicating the number of instances of said substituent can vary, then when the number of said instances is greater than 1, each instance is independently selected from the group of radicals defined for the substituent. Further, when the subscript indicates a range, e.g., (R^{d})ᵢ₋ⱼ, then the number of substituent instances may be selected from the integers between i and j inclusive.
“—CHC(O)O(CH₂)ₘ” means

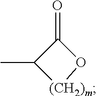

“—CHO(CH₂)ₙ” means

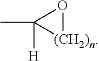

When a group contains a substituent which can be hydrogen, for example R¹⁵ or R³⁴, then, when this substituent is taken as hydrogen, it is recognized that this is equivalent to said group being unsubstituted.

Formula I compounds can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the present invention comprises fatty-amine salts of at least one Formula I compounds that may be present as a mixture of stepreoisomers, individual stereoisomers, or as an optically active form.

In one embodiment, agriculturally suitable salts of the present invention are salts formed by contact of at least one Formula I compound with long-chain fatty amines such that the derived salts retain sufficient water solubility for bioavailability and thus herbicidal efficacy and that the counterions of the salts are suitable for use in agriculture. One skilled in the art recognizes that because in the environment and under physiological conditions salts of the present invention are in equilibrium with their corresponding nonsalt forms, agriculturally suitable salts share the biological utility of the nonsalt forms.

In agriculturally suitable salts of Formula I compounds, R is CO₂H (including wherein R² is CO₂H) formed with long-chain fatty amines.

Long-chain fatty amines of the present invention have at least 10-carbon chain length. A preferred carbon chain length of the long-chain fatty acids is 10-25 carbons. Generally the long-chain fatty amines are liquid at room temperature. However, solid long-chain fatty amines that can be melted at elevated temperature and/or dissolved in a solvent or solvents can also be used. The long-chain fatty amines of the present invention also include linear or branched amines, and substituted or unsubstituted amines.

Solvents used for the neutralization reaction, described infra, include ethylene glycol monomethyl ether, ethylene glycol monoethyl ether, ethylene glycol monopropyl ether, ethylene glycol monobutyl ether, ethylene glycol monomethyl ether acetate, ethylene glycol monoethyl ether acetate, ethylene glycol monohexyl ether, ethylene glycol mono-2-ethylhexyl ether, ethylene glycol monophenyl ether, diethylene glycol monomethyl ether, diethylene glycol monoethyl ether, diethylene glycol monopropyl ether, diethylene glycol monobutyl ether, ethylene glycol monobutyl ether acetate, diethylene glycol monoethyl ether acetate, diethylene glycol monobutyl ether acetate, propylene glycol monomethyl ether, propylene glycol monopropyl ether, propylene glycol monobutyl ether, propylene glycol monophenyl ether, dipropylene glycol monomethyl ether, and propylene glycol monomethyl ether acetate.

Embodiments of the present invention include:

### Embodiment 1

A compound comprising a fatty-amine salt of Formula I wherein j is 0.

### Embodiment 2

A compound comprising a fatty-amine salt of Formula I wherein k is 0.

### Embodiment 3

A compound comprising a fatty-amine salt of Formula I wherein R¹⁵ is H.

### Embodiment 4

A compound of Embodiment 3 wherein R¹⁶ is H.

### Embodiment 5

A compound of comprising a fatty-amine salt Formula I wherein
R is CO₂R¹², CH₂OR¹³, CH(OR⁴⁶)(OR⁴⁷), CHO, C(═NOR¹⁴)H, C(═NNR⁴⁸R⁴⁹)H, C(═O)N(R¹⁸)R¹⁹, C(═S)OR⁵⁰, C(═O)SR⁵¹, C(═S)SR⁵² or C(═NR⁵³)YR⁵⁴;
R¹² is H, —CHC(O)O(CH₂)ₘ, —N═C(R⁵⁵)R⁵⁶; or a radical selected from C₁-C₁₄ alkyl, C₃-C₁₂ cycloalkyl, C₄-C₁₂ alkylcycloalklcyl, C₄-C₁₂ cycloalkylalkyl, C₂-C₁₄ alkenyl, C₂-C₁₄ alkynyl and phenyl, each radical optionally substituted with 1-3 R²⁷; or
R¹² is a divalent radical linking the carboxylic ester function CO₂R¹² of each of two pyrimidine ring systems of Formula I, the divalent radical selected from —CH₂—, —(CH₂)₂—, —(CH₂)₃— and —CH(CH₃)CH₂—;
R¹³ is H, C₁-C₁₀ alkyl optionally substituted with 1-3 R²⁸, or benzyl;
R¹⁴ is H, C₁-C₄ alkyl, C₁-C₄ haloalkyl or benzyl;
R¹⁸ is H, C₁-C₄ alkyl, hydroxy, C₁-C₄ alkoxy or S(O)₂R⁵⁷;
R¹⁹ is H or C₁-C₄ alkyl;
each R²⁷ is independently halogen, cyano, hydroxycarbonyl, C₂-C₄ alkoxycarbonyl, hydroxy, C₁-C₄ alkoxy, C₁-C₄ haloalkoxy, C₁-C₄ alkylthio, C₁-C₄ haloalkylthio, amino, C₁-C₄ alkylamino, C₂-C₄ dialkylamino, —CHO(CH₂)ₙ or phenyl optionally substituted with 1-3 R⁴⁴; or
two R²⁷ are taken together as —OC(O)O— or —O(C(R⁵⁸)(R⁵⁸))₁₋₂O—; or
two R²⁷ are taken together as an oxygen atom to form, with the carbon atom to which they are attached, a carbonyl moiety;
each R²⁸ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino; or
two R²⁸ are taken together as an oxygen atom to form, with the carbon atom to which they are attached, a carbonyl moiety;
each R⁴⁴ is independently halogen, C₁-C₄ alkyl, C₁-C₃ haloalkyl, hydroxy, C₁-C₄ alkoxy, C₁-C₃ haloalkoxy, C₁-C₃ alkylthio, C₁-C₃ haloalkylthio, amino, C₁-C₃ alkylamino, C₂-C₄ dialkylamino or nitro;
R⁴⁶ and R⁴⁷ are independently C₁-C₄ alkyl or C₁-C₃ haloalkyl; or
R⁴⁶ and R⁴⁷ are taken together as —CH₂CH₂—, —CH₂CH(CH₃)— or —(CH₂)₃—;
R⁴⁸ is H, C₁-C₄ alkyl, C₁-C₄ haloalkyl, C₂-C₄ alkylcarbonyl, C₂-C₄ alkoxycarbonyl or benzyl;
R⁴⁹ is H, C₁-C₄ alkyl or C₁-C₄ haloalkyl;
R⁵⁰, R⁵¹ and R⁵² are H; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl and C₂-C₁₄ alkynyl, each radical optionally substituted with 1-3 R²⁷;
Y is O, S or NR⁶¹;
R⁵³ is H, C₁-C₃ alkyl, C₁-C₃ haloalkyl, C₂-C₄ alkoxyalkyl, OH or C₁-C₃ alkoxy;
R⁵⁴ is C₁-C₃ alkyl, C₁-C₃ haloalkyl or C₂-C₄ alkoxyalkyl; or
R⁵³ and R⁵⁴ are taken together as —(CH₂)₂—, —CH₂CH(CH₃)— or —(CH₂)₃—;
R⁵⁵ and R⁵⁶ are independently C₁-C₄ alkyl;
R⁵⁷ is C₁-C₄ alkyl, C₁-C₃ haloalkyl or NR⁵⁹R⁶⁰;
each R⁵⁸ is independently selected from H and C₁-C₄ alkyl;
R⁵⁹ and R⁶⁰ are independently H or C₁-C₄ alkyl;
R⁶¹ is H, C₁-C₃ alkyl, C₁-C₃ haloalkyl or C₂-C₄ alkoxyalkyl;
m is an integer from 2 to 3; and
n is an integer from 1 to 4.

### Embodiment 6

A compound comprising a fatty-amine salt of Formula I wherein when R¹ is optionally substituted cyclopropyl, then R² is other than alkoxyalkyl or alkylthioalkyl.

### Embodiment 7

A comprising a fatty-amine salt compound of Formula I wherein R² is other than alkoxyalkyl or alkylthioalkyl.

### Embodiment 8

A compound of Embodiment 5 wherein
R² is CO₂R¹², CH₂OR¹³, CH(OR⁴⁶)(OR⁴⁷), CHO, C(═NOR¹⁴)H, C(═NNR⁴⁸R⁴⁹)H, (O)ⱼC(R¹⁵)(R¹⁶)CO₂R¹⁷, C(═O)N(R¹⁸)R¹⁹, C(═S)OR⁵⁰, C(═O)SR⁵¹, C(═S)SR⁵² or C(═NR⁵³)_{y}R⁵⁴;
R¹⁷ is C₁-C₁₀ alkyl optionally substituted with 1-3 R²⁹, or benzyl; and
each R²⁹ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino.

### Embodiment 9

A compound of Embodiment 8 wherein when R³ is CH₂OR¹³, then R¹³ is other than alkyl.

### Embodiment 10

A compound of Embodiment 8 wherein when R³ is CH₂OR¹³, then R¹³ is other than optionally substituted alkyl.

### Embodiment 11

A compound of Embodiment 8 wherein R³ is other than CH₂OR¹³.

### Embodiment 12

A compound of Embodiment 8 wherein j is 0.

### Embodiment 13

A compound of Embodiment 12 wherein R² is CO₂R¹², CH₂OR¹³, CHO or CH₂CO₂R¹⁷.

### Embodiment 14

A compound of Embodiment 13 wherein R² is CO₂R¹².

### Embodiment 15

A compound of Embodiment 14 wherein R¹² is H, C₁-C₈ alkyl or C₁ alkyl substituted with phenyl optionally substituted with 1-3 R⁴⁴.

### Embodiment 16

A compound of Embodiment 15 wherein R¹² is H, C₁-C₄ alkyl or C₁ alkyl substituted with phenyl optionally substituted with 1-3 R⁴⁴.

### Embodiment 17

A compound of Embodiment 16 wherein R¹² is H, C₁-C₄ alkyl or benzyl.

### Embodiment 18

A compound comprising a fatty-amine salt of Formula I wherein R² is CO₂H, an agriculturally suitable salt or an ester or thioester derivative thereof.

### Embodiment 19

A compound of Embodiment 18 wherein R² is CO₂H, an agriculturally suitable salt or an ester derivative thereof.

### Embodiment 20

A compound comprising a fatty-amine salt of Formula I wherein R¹ is cyclopropyl optionally substituted with 1-5 R⁵.

### Embodiment 21

A compound comprising a fatty-amine salt of Formula I wherein R¹ is isopropyl optionally substituted with 1-5 R⁶.

### Embodiment 22

A compound comprising a fatty-amine salt of Formula I wherein R¹ is phenyl optionally substituted with 1-3 R⁷.

### Embodiment 23

A compound comprising a fatty-amine salt of Formula I wherein R¹ is cyclopropyl optionally substituted with 1-5 R⁵ or isopropyl optionally substituted with 1-5 R⁶.

### Embodiment 24

A compound comprising a fatty-amine salt of Formula I wherein R¹ is cyclopropyl optionally substituted with 1-5 R⁵ or phenyl optionally substituted with 1-3 R⁷.

### Embodiment 25

A compound comprising a fatty-amine salt of Formula I wherein R¹ is isopropyl optionally substituted with 1-5 R⁶ or phenyl optionally substituted with 1-3 R⁷.

### Embodiment 26

A compound comprising a fatty-amine salt of Formula I wherein R¹ is other than cyclopropyl.

### Embodiment 27

A compound comprising a fatty-amine salt of Formula I wherein R¹ is cyclopropyl optionally substituted with 1-2 R⁶ or phenyl optionally substituted with 1-3 R⁷.

### Embodiment 28

A compound of Embodiment 27 wherein R¹ is cyclopropyl optionally substituted with 1-2 R⁶.

### Embodiment 29

A compound of Embodiment 27 wherein R¹ is cyclopropyl or phenyl optionally substituted with 1-3 R⁷.

### Embodiment 30

A compound of Embodiment 28 wherein R¹ is cyclopropyl.

### Embodiment 31

A compound of Embodiment 27 wherein R¹ is phenyl optionally substituted with 1-3 R⁷.

### Embodiment 32

A compound of Embodiment 27 wherein R¹ is cyclopropyl or phenyl substituted with a R⁷ radical in the para position and optionally with 1-2 R⁷ in other positions.

### Embodiment 33

A compound of Embodiment 32 wherein R¹ is cyclopropyl or phenyl substituted with a halogen, methyl or methoxy radical in the para position and optionally with 1-2 radicals selected from halogen and methyl in other positions.

### Embodiment 34

A compound of Embodiment 33 wherein R¹ is cyclopropyl or phenyl substituted with a halogen radical in the para position and optionally with 1-2 radicals selected from halogen and methyl in other positions.

### Embodiment 35

A compound of Embodiment 34 wherein R¹ is cyclopropyl or phenyl substituted with a Br or Cl radical in the para position and optionally with 1-2 radicals selected from halogen and methyl in other positions.

### Embodiment 36

A compound of Embodiment 35 wherein R¹ is phenyl substituted with a Br or Cl radical in the para position and optionally with 1-2 radicals selected from halogen and methyl in other positions.

### Embodiment 37

A compound of Embodiment 35 wherein R¹ is cyclopropyl or phenyl substituted with a Br or Cl radical in the para position.

### Embodiment 38

A compound of Embodiment 37 wherein R¹ is phenyl substituted with a Br or Cl radical in the para position.

### Embodiment 39

A compound comprising a fatty-amine salt of Formula I wherein R⁷ is other than cyano.

### Embodiment 40

A compound comprising a fatty-amine salt of Formula I wherein R⁷ selected from other than optionally substituted phenyl, phenoxy and 5- and 6-membered heteroaromatic rings.

### Embodiment 41

A compound comprising a fatty-amine salt of Formula I wherein each R⁷ is independently selected from halogen, C₁-C₂ alkyl, C₁-C₂ haloalkyl, C₁-C₂ alkoxy or C₁-C₂ haloalkoxy; or two adjacent R⁷ are taken together as —OCH₂O—, —CH₂CH₂O—, —OCH(CH₃)O—, —OC(CH₃)₂O—, —OCF₂O—, —CF₂CF₂O—, —OCF₂CF₂O— or —CH═CH—CH═CH—.

### Embodiment 42

A compound of Embodiment 41 wherein each R⁷ is independently selected from halogen, C₁-C₂ alkyl, C₁-C₂ haloalkyl, C₁-C₂ alkoxy or C₁₋₂ haloalkoxy; or two adjacent R⁷ are taken together as —OCH₂O—, —CH₂CH₂O—, —OCH(CH₃)O— or —OCF₂O—.

### Embodiment 43

A compound of Embodiment 42 wherein each R⁷ is independently selected from halogen, C₁-C₂ alkyl, C₁ fluoroalkyl, C₁-C₂ alkoxy or C₁ fluoroalkoxy.

### Embodiment 44

A compound comprising a fatty-amine salt of Formula I wherein each R⁷ is independently selected from halogen, methyl and methoxy.

### Embodiment 45

A compound of Embodiment 44 wherein each R⁷ is independently selected from halogen and methyl.

### Embodiment 46

A compound of Embodiment 45 wherein each R⁷ is independently selected from F, Cl and Br.

### Embodiment 47

A compound of Embodiment 46 wherein each R⁷ is independently selected from Cl and Br.

### Embodiment 48

A compound comprising a fatty-amine salt of Formula I wherein R³ is other than cyano.

### Embodiment 49

A compound comprising a fatty-amine salt of Formula I wherein R³ is other than nitro.

### Embodiment 50

A compound comprising a fatty-amine salt of Formula I wherein R³ is halogen, nitro, OR²⁰, SR²¹ or N(R²²)R²³.

### Embodiment 51

A compound of Embodiment 50 wherein R³ is halogen.

### Embodiment 52

A compound of Embodiment 51 wherein R³ is Br or Cl.

### Embodiment 53

A compound of Embodiment 52 wherein R³ is Cl.

### Embodiment 54

A compound comprising a fatty-amine salt of Formula I wherein R⁴ is —N(R²⁴)R²⁵.

### Embodiment 55

A compound comprising a fatty-amine salt of Formula I wherein R²⁴ is other than C₂-C₄ alkynyl optionally substituted with 1-2 R³².

### Embodiment 56

A compound of comprising a fatty-amine salt Formula I wherein R²⁴ is H, C(O)R³³ or C₁-C₄ alkyl optionally substituted with R³⁰; R²⁵ is H or C₁-C₂ alkyl; or R²⁴ and R²⁵ are taken together as ═C(R³⁹)N(R⁴⁰)R⁴¹.

### Embodiment 57

A compound of Embodiment 56 wherein R²⁴ is H, C(O)CH₃ or C₁-C₄ alkyl optionally substituted with R³⁰; and R²⁵ is H or C₁-C₂ alkyl.

### Embodiment 58

A compound of Embodiment 57 wherein R²⁴ and R²⁵ are independently H or methyl.

### Embodiment 59

A compound of Embodiment 58 wherein R²⁴ and R²⁵ are H.

### Embodiment 60

A compound comprising a fatty-amine salt of Formula I wherein R³⁰ is halogen, methoxy, C₁ fluoroalkoxy, methylthio, C₁ fluoroalkylthio, amino, methylamino, dimethylamino or methoxycarbonyl.

### Embodiment 61

A compound comprising a fatty-amine salt of Formula I wherein R³³ is H or C₁-C₃ alkyl.

### Embodiment 62

A compound of Embodiment 61 wherein R³³ is CH₃.

### Embodiment 63

A compound comprising a fatty-amine salt of Formula I wherein R³⁹ is H or C₁-C₂ alkyl.

### Embodiment 64

A compound comprising a fatty-amine salt of Formula I wherein R⁴⁰ and R⁴¹ are independently H or C₁-C₂ alkyl.

### Embodiment 65

A compound comprising a fatty-amine salt of Formula I wherein R³ is other than OH.

### Embodiment 66

A compound comprising a fatty-amine salt of Formula I wherein R³ is other than OR²⁰.

### Embodiment 67

A compound comprising a fatty-amine salt of Formula I wherein when j is 1, and R¹ is isopropyl substituted with at least one R⁶ being halogen, then R²⁴ and R²⁵ are each H.

### Embodiment 68

A compound comprising a fatty-amine salt of Formula I wherein when j is 1, R¹ is optionally substituted isopropyl, the R²⁴ and R²⁵ are each H.

### Embodiment 69

A compound comprising a fatty-amine salt of Formula I wherein when j is 1, then R²⁴ and R²⁵ are each H.

### Embodiment 70

A compound comprising a fatty-amine salt of Formula I wherein when j is 1, then R⁶ is other than halogen.

### Embodiment 71

A compound comprising a fatty-amine salt of Formula I wherein when j is 1, then R¹ is other than optionally substituted isopropyl.

### Embodiment 72

A compound comprising a fatty-amine salt of Formula I wherein when j is 1, then R¹ is cyclopropyl optionally substituted with 1-5 R⁵, isopropyl, or phenyl optionally substituted with 1-3 R⁷.

### Embodiment 73

A compound comprising a fatty-amine salt of Formula I wherein when j is 1, then R¹ is cyclopropyl, isopropyl, or phenyl optionally substituted with 1-3 R⁷.

### Embodiment 74

A compound comprising a fatty-amine salt of Formula I wherein when R¹ is phenyl optionally substituted with 1-3 R⁷ then R is other than cyano.

### Embodiment 75

A compound comprising a fatty-amine salt of Formula I wherein R is other than cyano.

### Embodiment 76

A compound of Embodiment 5 wherein when R¹ is phenyl optionally substituted with 1-3 R⁷ then R is CO₂R¹².

### Embodiment 77

A compound of Embodiment 5 wherein R is CO₂R¹².

### Embodiment 78

A compound of Embodiment 8 wherein when R¹ is phenyl optionally substituted with 1-3 R⁷ then R² is CO₂R¹².

### Embodiment 79

A compound of Embodiment 8 wherein R² is CO₂R¹².

### Embodiment 80

A compound comprising a fatty-amine salt of Formula I wherein when R¹ is phenyl optionally substituted with 1-3 R⁷ then R²⁴ is H, C(═O)R³³, nitro, OR³⁴, S(O)₂R³⁵ or N(R³⁶)R³⁷, and R²⁵ is H or C(═O)R³³.

### Embodiment 81

A compound comprising a fatty-amine salt of Formula I wherein when R¹ is phenyl optionally substituted with 1-3 R⁷ then R²⁴ and R²⁵ are each H.

### Embodiment 82

A compound comprising a fatty-amine salt of Formula I wherein R²⁴ is H, C(═O)R³³, nitro, OR³⁴, S(O)₂R³⁵ or N(R³⁶)R³⁷, and R²⁵ is H or C(═O)R³³.

### Embodiment 83

A compound comprising a fatty-amine salt of Formula I wherein R²⁴ and R²⁵ are each H.

### Embodiment 84

A compound comprising a fatty-amine salt of Formula I wherein when R¹ is cyclopropyl or isopropyl optionally substituted with 1-5 R⁶, then R is other than C(═W¹)N(R^{b1})S(O)₂—R^{cd} wherein W comprises at least one atom; R^{b1} comprises at least one atom and Red comprises at least one atom.

### Embodiment 85

A compound comprising a fatty-amine salt of Formula I wherein when R¹ is cyclopropyl optionally substituted with 1-5 R⁵ or isopropyl optionally substituted with 1-5 R⁶, then R is other than C(═W¹)N(R^{b1})S(O)₂—R^{d} wherein W comprises at least one atom; R^{b1} comprises at least one atom and Rcd comprises at least one atom.

### Embodiment 86

A compound comprising a fatty-amine salt of Formula I wherein R is other than C(═W¹)N(R^{b1})S(O)₂—R^{cd} wherein W comprises at least one atom; R^{b1} comprises at least one atom and Red comprises at least one atom.

### Embodiment 87

A compound of Embodiment 5 wherein R¹⁸ is H, C₁-C₄ alkyl, hydroxy or C₁-C₄ alkoxy.

### Embodiment 88

A compound of Embodiment 8 wherein R¹⁸ is H, C₁-C₄ alkyl, hydroxy or C₁-C₄ alkoxy.

### Embodiment 89

A compound comprising a fatty-amine salt of Formula I wherein each R⁵ and R⁶ is independently halogen, C₁-C₂ alkyl or C₁-C₂ haloalkyl.

### Embodiment 90

A compound of Formula I wherein R¹⁵ is H, halogen, C₁-C₄ alkyl, C₁-C₄ haloalkyl, hydroxy, C₁-C₄ alkoxy or C₂-C₄ alkylcarbonyloxy.

### Embodiment 91

A compound comprising a fatty-amine salt of Formula I wherein R¹⁶ is H, halogen, C₁-C₄ alkyl or C₁-C₄ haloalkyl.

### Embodiment 92

A compound comprising a fatty-amine salt of Formula I wherein R²⁴ is H, C₁-C₄ alkyl optionally substituted with 1-2 R³⁰, C₂-C₄ alkenyl optionally substituted with 1-2 R³¹, or C₂-C₄ alkynyl optionally substituted with 1-2 R³²; or R²⁴ is C(═O)R³³, nitro, OR³⁴, S(O)₂R³⁵ or N(R³⁶)R³⁷.

### Embodiment 93

A compound comprising a fatty-amine salt of Formula I wherein each R³³ is independently H,
C₁-C₄ alkyl, C₁-C₃ haloalkyl, C₁-C₄ alkoxy, phenoxy or benzyloxy.

### Embodiment 94

A compound comprising a fatty-amine salt of Formula I wherein R³⁴ is H, C₁₋₄alkyl or C₁-C₃ haloalkyl.

### Embodiment 95

A compound comprising a fatty-amine salt of Formula I wherein R³⁶ is H or C₁-C₄ alkyl.

### Embodiment 96

A compound of Embodiment 5 wherein R¹² is H; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl and C₂-C₁₄ alkynyl, each radical optionally substituted with 1-3 R²⁷; or —N═C(R⁵⁵)R⁵⁶.

### Embodiment 97

A compound of Embodiment 5 wherein each R²⁷ is independently halogen, hydroxycarbonyl, C₂-C₄ alkoxycarbonyl, hydroxy, C₁-C₄ alkoxy, C₁-C₄ haloalkoxy, C₁-C₄ alkylthio, C₁-C₄ haloalkylthio, amino, C₁-C₄ alkylamino, C₂-C₄ dialkylamino, —CHO(CH₂)ₙ or phenyl optionally substituted with 1-3 R⁴⁴; or two R²⁷ are taken together as —OC(O)O— or —O(C(R⁵⁸)(R⁵⁸))₁₋₂O—; or two R²⁷ are taken together as an oxygen atom to form, with the carbon atom to which they are attached, a carbonyl moiety.

### Embodiment 98

A compound of Embodiment 5 wherein R⁵³ is H, C₁-C₃ alkyl, C₁-C₃ haloalkyl or C₂-C₄ alkoxyalkyl.

Combinations of Embodiments 1-98 are illustrated by:

### Embodiment A

A compound comprising a fatty-amine salt of Formula I wherein
R² is CO₂R¹², CH₂OR¹³, CH(OR⁴⁶)(OR⁴⁷), CHO, C(═NOR¹⁴)H, C(═NNR⁴⁸R⁴⁹)H, (O)ⱼC(R¹⁵)(R¹⁶)CO₂R¹⁷, C(═O)N(R¹⁸)R¹⁹, C(═S)OR⁵⁰, C(═O)SR⁵¹, C(═S)SR⁵² or C(═NR⁵³)YR⁵⁴;
R¹² is H, —CHIC(O)O(CH₂)ₘ₄, —N═C(R⁵⁵)R⁵⁶; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl, C₂-C₁₄ alkynyl and phenyl, each radical optionally substituted with 1-3 R²⁷; or
R¹² is a divalent radical linking the carboxylic ester function CO₂R¹² of each of two pyrimidine ring systems of Formula I, the divalent radical selected from —CH₂—, —(CH₂)₂—, —(CH₂)₃— and —CH(CH₃)CH₂—;
R¹³ is H, C₁-C₁₀ alkyl optionally substituted with 1-3 R²⁸, or benzyl;
R¹⁴ is H, C₁-C₄ alkyl, C₁-C₄ haloalkyl or benzyl;
R¹⁷ is C₁-C₁₀ alkyl optionally substituted with 1-3 R²⁹, or benzyl;
R¹⁸ is H, C₁-C₄ alkyl, hydroxy, C₁-C₄ alkoxy or S(O)₂R⁵⁷;
R¹⁹ is H or C₁-C₄ alkyl;
each R²⁷ is independently halogen, cyano, hydroxycarbonyl, C₂-C₄ alkoxycarbonyl, hydroxy, C₁-C₄ alkoxy, C₁-C₄ haloalkoxy, C₁-C₄ alkylthio, C₁-C₄ haloalkylthio, amino, C₁-C₄ alkylamino, C₂-C₄ dialkylamino, —CHO(CH₂)ₙ or phenyl optionally substituted with 1-3 R⁴⁴; or
two R²⁷ are taken together as —OC(O)O— or —O(C(R⁵⁸)(R⁵⁸))₁₋₂O—; or
two R²⁷ are taken together as an oxygen atom to form, with the carbon atom to which they are attached, a carbonyl moiety;
each R²⁸ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino; or
two R²⁸ are taken together as an oxygen atom to form, with the carbon atom to which they are attached, a carbonyl moiety;
each R²⁹ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino;
each R⁴⁴ is independently halogen, C₁-C₄ alkyl, C₁-C₃ haloalkyl, hydroxy, C₁-C₄ alkoxy, C₁-C₃ haloalkoxy, C₁-C₃ alkylthio, C₁-C₃ haloalkylthio, amino, C₁-C₃ alkylamino, C₂-C₄ dialkylamino or nitro;
R⁴⁶ and R⁴⁷ are independently C₁-C₄ alkyl or C₁-C₃ haloalkyl; or
R⁴⁶ and R⁴⁷ are taken together as —CH₂CH₂—, —CH₂CH(CH₃)— or —(CH₂)₃—;
R⁴⁸ is H, C₁-C₄ alkyl, C₁-C₄ haloalkyl, C₂-C₄ alkylcarbonyl, C₂-C₄ alkoxycarbonyl or benzyl;
R⁴⁹ is H, C₁-C₄ alkyl or C₁-C₄ haloalkyl;
R⁵⁰, R⁵¹ and R⁵² are H; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl and C₂-C₁₄ alkynyl, each radical optionally substituted with 1-3 R²⁷;
Y is O, S or NR⁶¹;
R⁵³ is H, C₁-C₃ alkyl, C₁-C₃ haloalkyl, C₂-C₄ alkoxyalkyl, OH or C₁-C₃ alkoxy;
R⁵⁴ is C₁-C₃ alkyl, C₁-C₃ haloalkyl or C₂-C₄ alkoxyalkyl; or
R⁵³ and R⁵⁴ are taken together as —(CH₂)₂—, —CH₂CH(CH₃)— or —(CH₂)₃—;
R⁵⁵ and R⁵⁶ are independently C₁-C₄ alkyl;
R⁵⁷ is C₁-C₄ alkyl, C₁₋₃ haloalkyl or NR⁵⁹R⁶⁰;
each R⁵⁸ is independently selected from H and C₁-C₄ alkyl;
R⁵⁹ and R⁶⁰ are independently H or C₁-C₄ alkyl;
R⁶¹ is H, C₁-C₃ alkyl, C₁-C₃ haloalkyl or C₂-C₄ alkoxyalkyl;
m is an integer from 2 to 3; and
n is an integer from 1 to 4.

### Embodiment B

A compound of Embodiment A wherein R³ is halogen.

### Embodiment C

A compound of Embodiment B wherein R¹ is cyclopropyl or phenyl substituted with a halogen, methyl or methoxy radical in the para position and optionally with 1-2 radicals selected from halogen and methyl in other positions; and R⁴ is —N(R²⁴)R²⁵.

### Embodiment D

A compound of Embodiment C wherein R² is CO₂R¹², CH₂OR¹³, CHO or CH₂CO₂R¹⁷.

### Embodiment E

A compound of Embodiment D wherein R²⁴ is H, C(O)R³³ or C₁-C₄ alkyl optionally substituted with R³⁰; R²⁵ is H or C₁-C₂ alkyl; or R²⁴ and R²⁵ are taken together as ═C(R³⁹)N(R⁴⁰)R⁴¹.

### Embodiment F

A compound of Embodiment E wherein R² is CO₂R¹²; and R²⁴ and R²⁵ are H.

### Embodiment G

A compound of Embodiment F wherein R¹² is H, C₁-C₄ alkyl or benzyl.

Specific embodiments include compounds of comprising a fatty-amine salt Formula I selected from the group consisting of:
methyl 6-amino-5-bromo-2-cyclopropyl-4-pyrimidinecarboxylate,
ethyl 6-amino-5-bromo-2-cyclopropyl-4-pyrimidinecarboxylate,
phenylmethyl 6-amino-5-bromo-2-cyclopropyl-4-pyrimidinecarboxylate,
6-amino-5-bromo-2-cyclopropyl-4-pyrimidinecarboxylic acid monosodium salt,
methyl 6-amino-5-chloro-2-cyclopropyl-4-pyrimidinecarboxylate,
phenylmethyl 6-amino-5-chloro-2-cyclopropyl-4-pyrimidinecarboxylate,
6-amino-5-chloro-2-cyclopropyl-4-pyrimidinecarboxylic acid monosodium salt,
ethyl 6-amino-5-chloro-2-cyclopropyl-4-pyrimidinecarboxylate,
methyl 6-amino-5-chloro-2-(4-chlorophenyl)-4-pyrimidinecarboxylate,
ethyl 6-amino-5-chloro-2-(4-chlorophenyl)-4-pyrimidinecarboxylate,
6-amino-5-chloro-2-(4-chlorophenyl)-4-pyrimidinecarboxylic acid,
ethyl 6-amino-2-(4-bromophenyl)-5-chloro-4-pyrimidinecarboxylate,
methyl 6-amino-2-(4-bromophenyl)-5-chloro-4-pyrimidinecarboxylate, and
6-amino-2-(4-bromophenyl)-5-chloro-4-pyrimidinecarboxylic acid.

Also noteworthy as embodiments are herbicidal compositions of the present invention comprising the compounds of embodiments described above.

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above.

Of note is a compound of Formula I, including all geometric and stereoisomers, N-oxides or agriculturally suitable salts thereof, agricultural compositions containing them and their use as herbicides wherein R² is CO₂R¹², CH₂OR¹³, CHO, C(═NOR¹⁴)H, C(R¹⁵)(R¹⁶)CO₂R¹⁷ or C(═O)N(R¹⁸)R¹⁹; each R⁷ is independently halogen, C₁-C₄ alkyl, C₁-C₃ haloalkyl, C₁-C₃ alkoxy, C₁-C₃ haloalkoxy, C₁-C₃ alkylthio or C₁-C₃ haloalkylthio; R¹² is H; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl and C₂-C₁₄ alkynyl, each radical optionally substituted with 1-3 R²⁷; R¹³ is H, C₁-C₁₀ alkyl optionally substituted with 1-3 R²⁸ or benzyl; R¹⁴ is H, C₁-C₄ alkyl or C₁-C₄ haloalkyl; R¹⁵ and R¹⁶ are independently H, halogen, C₁-C₄ alkyl, C₁-C₄ haloalkyl, hydroxy or C₁-C₄ alkoxy; R¹⁷ is C₁-C₁₀ alkyl optionally substituted with 1-3 R²⁹ or benzyl; R¹⁸ and R¹⁹ are independently H or C₁-C₄ alkyl; each R²⁷ is independently halogen, hydroxycarbonyl, C₂-C₄ alkoxycarbonyl, hydroxy, C₁-C₄ alkoxy, C₁-C₄ haloalkoxy, C₁-C₄ alkylthio, C₁-C₄ haloalkylthio, amino, C₁-C₄ alkylamino, C₂₋₄ dialkylamino, —CHO(CH₂)ₙ or phenyl optionally substituted with 1-3 R⁴⁴; or two R²⁷ are taken together with the carbon atom to which they are attached to form a carbonyl moiety; each R²⁸ and R²⁹ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino; each R³⁰, R³¹ and R³² is independently halogen, hydroxy, C₁-C₄ alkoxy, C₁-C₄ haloalkoxy, C₁-C₄ alkylthio, C₁-C₄ haloalkylthio, amino, C₁-C₄ alkylamino, C₂-C₄ dialkylamino or C₂-C₄ alkoxycarbonyl; each R³⁸ is independently halogen, C₁-C₃ alkyl, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl; each R⁴⁴ is independently halogen, C₁-C₄ alkyl, C₁-C₃ haloalkyl, hydroxy, C₁-C₄ alkoxy, C₁-C₃ haloalkoxy, C₁-C₄ alkylthio, C₁-C₃ haloalkylthio, amino, C₁-C₃ alkylamino, C₂-C₄ dialkylamino or nitro; m is an integer from 2 to 5; and n is an integer from 1 to 4. Also of note is a compound of Formula I, including all geometric and stereoisomers, N-oxides or agriculturally suitable salts thereof, agricultural compositions containing them and their use as herbicides wherein each R⁵ and R⁶ is independently halogen, C₁-C₂ alkyl or C₁-C₂ haloalkyl; R¹⁵ is H, halogen, C₁-C₄ alkyl, C₁-C₄ haloalkyl, hydroxy, C₁-C₄ alkoxy or C₂-C₄ alkylcarbonyloxy; R¹⁶ is H, halogen, C₁-C₄ alkyl or C₁-C₄ haloalkyl; R²⁴ is H, C₁-C₄ alkyl optionally substituted with 1-2 R³⁰, C₂-C₄ alkenyl optionally substituted with 1-2 R³¹, or C₂-C₄ alkynyl optionally substituted with 1-2 R³²; or R²⁴ is C(═O)R³³, nitro, OR³⁴, S(O)₂R³⁵ or N(R³⁶)R³⁷; each R³³ is independently H, C₁-C₄ alkyl, C₁-C₃ haloalkyl, C₁-C₄ alkoxy, phenoxy or benzyloxy; R³⁴ is H, C₁-C₄ alkyl, C₁-C₃ haloalkyl; and R³⁶ is H or C₁-C₄ alkyl.

Fatty-amine salt of the present invention are prepared by a weak acid-weak base neutralization reaction, wherein the amine functional group of the long-chain fatty amine reacts with the carboxyl group in the R² position of the compound of Formula I.

In one embodiment, the neutralization reaction is conducted in such manner that the reactants in a solvent undergo some form of shear. Shear can be induced, for example, by mixing. Various methods for mixing the reactants can be used. Mixing techniques are described in, for example, Perry's Chemical Engineer's Handbook (Ref. Formula I compounds in particulate form can be mixed with at least one long-chain fatty amine under high shear conditions. In one embodiment, the particulates are wetted by the solvent and/or the long-chain fatty acid. In another embodiment, the particulate contact angle in comparison with the solvent and/or the long-chain fatty acids is such that the particulates remain unwetted. In one embodiment, the neutralization reaction between the Formula I compound and the long-chain fatty acid is exothermic. In another embodiment, the neutralization reaction between the Formula I compound and the long-chain fatty acid is endothermic.

In one embodiment, particulates of a Formula I compound are prewetted to reduce the surface tension, which, it is theorized, helps to increase available surface area of the particulates of Formula I compound. This invention is not bound by any theory, however. Prewetted particulates of Formula I compound may be neutralized by reaction with long-chain fatty acid with lower shear than otherwise required. In one embodiment, the total heat of the system is more than the exothermic heat released by the reaction. In a preferred embodiment, the additional heat in the system is substantially shear-induced.

In one embodiment, the neutralization reaction is carried out without shear. In another embodiment, the neutralization reaction is carried out without shear but at a higher temperature.

In another embodiment, the neutralization reaction, an endothermic reaction, is carried out without shear but at a higher temperature.

In a preferred embodiment, the Formula I compound is 6-amino-5-chloro-2-cyclopropylpyrimidine-4-carboxylic acid. In another preferred embodiment, the long-chain fatty amine is oleylamine. In a further preferred embodiment, the solvent is dipropylene glycol monomethyl ether.

Preparation of several compounds of Formula I are described in U.S. Pat. No. 7,863,220, which is incorporated by reference herein.

One skilled in the art will also recognize that compounds of Formula I and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “ddd” means doublet of doublets of doublets, “dt” means doublet of triplets, “dq” means doublet of quartets, “br s” means broad singlet, “br d” means broad doublet.

### Example 1

### Preparation of 6-amino-5-chloro-2-cyclopropyl-4-primidinecarboxylic acid (Compound 135)

### Step A1: Preparation of 2-cyclopropyl-1,6-dihydro-6-oxo-4-pyrimidinecarboxylic acid

To a mixture of diethyl oxalacetate sodium salt (150 g, 714 mmol) in methanol (300 mL) and water (150 mL) warmed to 30° C. was added 50% aqueous sodium hydroxide (56 g, 700 mmol) in water (60 mL) over 30 minutes, over which time the temperature remained at 25-30° C. and the pH at 11-12. Then the stirred mixture was heated for an additional 30 min at 35° C. To this mixture was added cyclopropanecarboximidamide monohydrochloride (64 g, 530 mol) in portions over 15 minutes. The orange solution was heated to 50° C. over 30 minutes and held at that temperature for 3 h. The reaction mixture was cooled to 35° C., and concentrated hydrochloric acid (ca. 70 g, 0.7 mol) was added gradually (resulting in foaming) over 30 minutes at 30-40° C. until the pH was about 1.5-2.5. The mixture was concentrated with a rotary evaporator at 35-40° C. to remove alcohols, stirred for 3-4 h at 25° C. to complete crystallization of the product. After the mixture was cooled to 0° C. the solid was collected by filtration. The solid was washed with water (2×60 mL), suction-dried, and then dried in a vacuum-oven at 60° C. to afford the title compound as a beige solid (ca. 60 g).

¹H NMR (DMSO-d₆) δ 6.58 (s, 1H), 1.95 (m, 1H), 1.0 (m, 4H).

### Step A2: Another preparation of 2-cyclopropyl-1,6-dihydro-6-oxo-4-pyrimidinecarboxylic acid

To a mixture of diethyl oxalacetate sodium salt (210 g, 950 mmol) in methanol (500 mL) and water (400 mL) was added 50% aqueous sodium hydroxide (80 g, 1.0 mol) in water (60 mL) over 30 minutes, over which time the temperature remained at 25-30° C. and the pH at 11-12. Then the stirred mixture was heated for an additional 30 min at 30° C. To this mixture was added cyclopropanecarboximidamide monohydrochloride (110 g, 910 mol). The orange solution was heated to 50° C. over 30 minutes and held at that temperature for 5 h. The reaction mixture was cooled to 30° C. and concentrated to half volume at reduced pressure at 35-40° C. and concentrated hydrochloric acid (140 g, 1.4 mol) was added gradually (resulting in foaming) over 30 minutes at 25-30° C. until the pH was about 1-2. The mixture was stirred at 5° C. for 1 h to complete crystallization of the product. After the mixture was cooled to 0° C. the solid was collected by filtration. The solid was washed with water (3×60 mL), suction-dried, and then dried in a vacuum-oven at 70° C. to afford the title compound as a beige solid (100 g); m.p. 235-236° C. (dec.).

¹H NMR (DMSO-d₆) δ 6.58 (s, 1H), 1.95 (m, 1H), 1.0 (m, 4H).

Step B1: Preparation of 5-chloro-2-cyclopropyl-1,6-dihydro-6-oxo-4-pyrimidinecarboxylic acid

To a mixture of 2-cyclopropyl-1,6-dihydro-6-oxo-4-pyrimidinecarboxylic acid (i.e. the product of Step A1 or A2) (9.2 g, 52 mmol) in water (30 mL) and concentrated hydrochloric acid (22 g, 220 mmol) at 15° C. was added dropwise aqueous sodium hypochlorite solution (11%, 40 g, 59 mmol) over 15 minutes so that with cooling the reaction mixture was maintained at 15-20° C. The mixture was then held at 20-25° C. for 1 h. Solid sodium bisulfite (ca. 2 g) was added, and then aqueous sodium hydroxide solution (50%, 8 g, 0.10 mol) was added dropwise so that with cooling the reaction mixture was maintained at about 25° C. The mixture was cooled to 10° C., and the suspended product was isolated by filtration and washed with a minimum amount of cold water. The product was then dried to constant weight in vacuum-oven at 50° C. to afford the title compound (7.5 g).

¹H NMR (DMSO-d₆) δ 13.4 (br s, 1H), 1.95 (m, 1H), 1.0 (m, 4H).

### Step B2: Another preparation of 5-chloro-2-cyclopropyl-1,6-dihydro-6-oxo-4-pyrimidinecarboxylic acid

To a mixture of 2-cyclopropyl-1,6-dihydro-6-oxo-4-pyrimidinecarboxylic acid (i.e. the product of Step A1 or A2) (184 g, 1.02 mol) in water (45 mL) and concentrated hydrochloric acid (292 g, 3 mol) at 8-12° C. was added dropwise aqueous sodium hypochlorite solution (8.4%, 1.02 kg, 1.15 mol) over 2 h so that with cooling the reaction mixture was maintained at 8-10° C. The mixture was then held at 10-12° C. for 1 h and the conversion was monitored by HPLC. When less than 5% of the starting material remained solid sodium bisulfite was added until a negative KI starch paper test was obtained. The mixture was cooled to 5° C., and the suspended product was isolated by filtration and washed with a minimum amount of cold water. The product was then dried to constant weight in vacuum-oven at 50° C. to afford the title compound (194 g); m.p. 189-190° C.

¹H NMR (DMSO-d₆) δ 13.4 (br s, 1H), 1.95 (m, 1H), 1.0 (m, 4H).

### Step C1: Preparation of 5,6-dichloro-2-cyclopropyl-4-pyrimidinecarboxylic acid

Phosphorus oxychloride (14 mL, 23 g, 0.15 mol) and 5-chloro-2-cyclopropyl-1,6-dihydro-6-oxo-4-pyrimidinecarboxylic acid (i.e. the product of Step B or B2) (75 g, 300 mmol) were combined and heated at 85° C. for 3 h. The reaction mixture was cooled to 30° C. and added over 30 minutes to a mixture of acetonitrile (50 mL) and ice water (80 mL), with the temperature maintained at 5-10° C. and the pH maintained in the range 1-3 by co-feeding aqueous ammonia (28%). The pH was adjusted to about 2, the mixture was concentrated at 25° C. with a rotary evaporator to remove acetonitrile, and the precipitated product was isolated by filtration and washed with water (2×25 mL). The solid was dried in a vacuum oven to afford the title compound (ca. 7.0 g).

¹H NMR (DMSO-d₆) δ 2.23 (m, 1H), 1.2 (m, 2H), 1.0 (m, 2H).

### Step C2: Another preparation of 5,6-dichloro-2-cyclopropyl-4-pyrimidinecarboxylic acid

Phosphorus oxychloride (200 mL, 328 g, 2.14 mol) and 5-chloro-2-cyclopropyl-1,6-dihydro-6-oxo-4-pyrimidinecarboxylic acid (i.e. the product of Step B1 or B2) (96.8 g, 451 mmol) were combined and heated at 90° C. for 5 h. The reaction mixture was cooled to 50-60° C. and concentrated at reduced pressure to half volume. After cooling to 30° C. the reaction mixture was added over 60 minutes to a mixture of t-butanol (200 mL) and water (300 mL), with the temperature maintained at 8-10° C. The mixture was seeded, water (300 mL) was added gradually at 10-15° C. and the mixture was stirred for 1 h. After cooling to 5° C. the precipitated product was isolated by filtration and washed with water (3×50 mL). The solid was dried in a vacuum oven to afford the title compound (93 g).

¹H NMR (DMSO-d₆) δ 2.23 (m, 1H), 1.2 (m, 2H), 1.0 (m, 2H).

### Step D1: Preparation of 6-amino-5-chloro-2-cyclopropyl-4-pyrimidinecarboxylic acid

A mixture of 5,6-dichloro-2-cyclopropyl-4-pyrimidinecarboxylic acid (i.e. the product of Step C1 or C2) (5.1 g, 22 mmol), water (30 mL) and aqueous ammonia (28%, 8 g, 130 mmol) was heated at 80° C. for 3 h. The solution was concentrated at 50° C. and 70 tort (9.3 kPa) pressure to about half volume to remove most of the excess ammonia. The resulting slurry was stirred at 20° C., acidified to pH 2 with aqueous hydrochloric acid, cooled to 5° C. and filtered. The isolated solid was dried in a vacuum oven to afford the title product (4.2 g), a compound of the present invention.

¹H NMR (DMSO-d₆) δ 13.4 (br s, 1H), 1.95 (m, 1H), 1.0 (m, 4H).

### Step D2: Another preparation of 6-amino-5-chloro-2-cyclopropyl-4-pyrimidinecarboxylic acid

A mixture of 5,6-dichloro-2-cyclopropyl-4-pyrimidinecarboxylic acid (i.e. the product of Step C1 or C2) (280 g, 1.2 mol), water (1.26 L) and aqueous ammonia (28%, 350 g, 5.76 mol) was heated at 80° C. for 5 h. The solution was concentrated at 50° C. and 70 torr (9.3 kPa) pressure to about half volume to remove most of the excess ammonia. The resulting slurry was stirred at 20° C., acidified to pH 1-2 with aqueous hydrochloric acid, cooled to 5° C. and filtered. The isolated solid was dried in a vacuum oven to afford the title product (270 g), a compound of the present invention.

¹H NMR (DMSO-d₆) δ 13.4 (br s, 1H), 1.95 (m, 1H), 1.0 (m, 4H).

### Example 2

### Preparation of the Liquid Intermediate of 6-amino-5-chloro-2-cycloprop lpyrimidine-4-carboxylic Acid Reacted with Oleylamine (MAT28 Liquid Intermediate) Under High Shear Conditions

The 6-amino-5-chloro-2-cyclopropylpyrimidine-4-carboxylic acid (MAT28) active ingredient is supplied as a fine technical powder which is 89.3% pure. In this embodiment, the active ingredient is applied to current base fertilizers by producing a 13% liquid intermediate which would be sprayed onto the base in the blender. The MAT28 liquid intermediate is formed via a weak acid-weak base neutralization reaction between the carboxylic acid group of the MAT28 molecule and the amine functional group of an oleylamine. This reaction forms a salt which, when dissolved in a glycol ether solvent, creates the desired liquid MUP. In the present embodiment of the invention the MAT28 MUP uses a high-shear mixing method. The reaction between MAT28 and oleylamine is slow at room temperature and under normal mixing conditions. However, the reaction time is considerably shortened when high shear mixing is utilized, making the creation of the liquid MUP possible in an acceptable time period.

### Manufacture of 13% MUP

The rate of application of MAT28 onto Northern fertilizer base is 0.065% which can be achieved by applying a 13% MAT28 liquid intermediate onto the base at a level of 0.5% liquid loading. The processes for manufacturing a 13% MAT28 liquid intermediate are outlined below.

### Hopper Discharge to Quadro High Shear Mixer

One bulk sack of the MAT28 technical powder per batch is fed to a powder hopper. This hopper discharges the technical powder to the cone on the top of the high-shear Quadro unit where it is sheared to facilitate the reaction between the MAT28 powder and oleylamine. The Quadro unit for manufacture of the liquid intermediate is the ZC3 model which handles approximately 7,200 lbs/hr. of the powder. The operating range for the MAT28 stream going into the Quadro is 65-130° F. due to the pour point and flash point of the oleylamine and glycol ether DPM solvent, respectively.

Oleylamine Feed Oleylamine is fed directly to the reaction vessel from a larger oleylamine storage tank. The

operating range for this stream is 65-130° F. due to the pour point of the oleylamine (59° F.) and the flash point of the glycol ether DPM solvent (167° F.).

### Glycol Ether DPM Feed

The glycol ether solvent is also fed directly to the reaction vessel from a storage tank. The operating range for this stream is also 65-130° F. based on the pour point of oleylamine and the flash point of the glycol ether solvent.

### Quadro Loop

Once the oleylamine and glycol ether are added to the reaction vessel and adequately mixed, the solution is cycled through a loop connecting the Quadro unit and the reaction vessel. Once the inlet pump and Quadro mixer are turned on the MAT28 powder is pulled into the high shear portion of the Quadro and sheared into the oleylamine and solvent mixture. In one embodiment, the entire reaction vessel is turned over approximately 6-8 times through this loop to completely react the MAT28 powder with the oleylamine. For this reason cooling capabilities are recommended for the reaction vessel as the use of high shear mixing introduces heat into the liquid during the cycling process. The recommended temperature range for this loop is 65-130° F. based on the pour point of the unreacted oleylamine (59° F.) and the flash point of the solvent (167° F.).

### Feed to Packaging

Once the reaction is complete (6-8 cycles through the loop), the pump and Quadro are shut off and the 13% MUP is emptied completely into the reaction vessel. The operating range for this stream is −4-130° F. since the finished MUP does not have the pour point restriction of the unreacted oleylamine.

### Composition

| Trade Name | Chemical Name | CAS# | Wt % | Purpose |
|---|---|---|---|---|
| Aminopyrachlor | aminopyrachlor | 858956-08-8 | 14.60% | Active |
| Technical | | | | |
| Dowanol DPM | dipropylene glycol | 34590-94-8 | 67.20% | Solvent |
| | monomethyl ether | | | |
| Armeen OL | oleylamine | 112-90-3 | 18.2% | Solvent |

### Calculation of Batch Size and Material Volumes, 13% MUP

For one embodiment, it is recommended that the MAT28 technical powder be received in bulk sacks weighing approximately 2,000 lbs. and that batches of the Northern concentration liquid intermediate (13% MAT28) be prepared based on the total weight of reactive MAT species contained in one bulk sack. The oleylamine and reactive MAT combination should be added such that an equal number of moles of each material are present in the reaction vessel. To do this, the purity of both the MAT28 technical powder and the oleylamine must be taken into account. The MAT28 technical is a combination of MAT28 and MAT26 powder with a purity approximately 89.3% (typical range of 89.3% to 100%1) and ˜0.60%, respectively. Oleylamine purity is characterized by the amine number which the supplier uses in order to calculate an equivalent molecular weight for each batch of the oleylamine. This equivalent molecular weight is used along with the purity of the MAT28 technical powder to arrive at a ratio of MAT28 technical to oleylamine in the reaction vessel. For reference, a sample calculation is also provided below.

Example: 2,000 lbs. MAT28 technical powder, 269 g/mol equivalent molecular weight oleylamine, 8% over-formulation factor on oleylamine.

### Reactive MAT Species Pure Weight:

$$\frac{0.905 lbs . Pure MAT 28}{lb . MAT 28 Technical lb .} + \frac{0.0060 lbs . Pure MAT 26}{MAT 28 Technical} = \frac{0.911 lbs of pure reacting MAT}{lb . MAT 28 Technical}$$

### Total Oleylamine Needed:

$$2 \text{,} 000 lb . MAT 28 Tech . \times \frac{0.911 lbs . Reactive MAT}{lb . MAT 28 Tech .} \times \frac{453.59 g . Reactive MAT}{lb . Reactive MAT} \times \frac{1 mol Reactive MAT}{213 g Reactive MAT} \times \frac{1 mol oleylamine}{1 mol Reactive MAT 28} \times \frac{269 equiv . g . oleylamine}{1 mol oleylamine} \times \frac{lb . oleylamine}{453.59 g . oleylamine} = 2 \text{,} 301.023 lb . oleylamine$$

### With an Over Formulation of 8% on Oleylamine:

2,301.023 lbs oleylamine×1.08 over-formulation factor=2,485.105 lbs. oleylamine needed

Ratio of MAT28 Technical to Oleylamine: 1:1.24

### Total Glycol Ether to be Added to Make a 13% Reactive MAT by Weight:

$$\underline{( 2 \text{,} 000 lb . \times 0.911 ) lb . Reactive MAT} \times \frac{1 lb . MUP}{0.13 lb . MAT 28} = 14 \text{,} 015.34 lbs . MUP total$$

$$14 \text{,} 015.34 lb . MUP - 2 \text{,} 000 lb . MAT 28 Tech . - 2 \text{,} 485.105 lb . Oleylamine = 9 \text{,} 530.254 lb . glycol ether DPM$$

### Example 3

### Preparation of the Liquid Intermediate of 6-amino-5-chloro-2-cyclopropylpyrimidine-4-Carboxylic Acid Reacted with Oleylamine (MAT28 Liquid Intermediate) Under Low- or Non-Shear Conditions without Solvent

The 6-amino-5-chloro-2-cyclopropylpyrimidine-4-carboxylic acid (MAT28) active ingredient is supplied as a fine technical powder which is 89.3% pure. In this embodiment, the active ingredient is applied to current base fertilizers by producing a 13% liquid intermediate which would be sprayed onto the base in the blender. The MAT28 liquid intermediate is formed via a weak acid-weak base neutralization reaction between the carboxylic acid group of the MAT28 molecule and the amine functional group of an oleylamine. In the present embodiment of the invention the MAT28 MUP uses a no-shear or a low-shear mixing method.

### Manufacture of 13% MUP

The rate of application of MAT28 onto Northern fertilizer base is 0.065% which can be achieved by applying a 13% MAT28 liquid intermediate onto the base at a level of 0.5% liquid loading. The processes for manufacturing a 13% MAT28 liquid intermediate are outlined below.

### Discharge to Reactor

One bulk sack of the MAT28 technical powder per batch is fed to a powder hopper. This hopper discharges the technical powder to the reactor. The operating range for the MAT28 stream going into the reactor is 65-130° F. due to the pour point and flash point of the oleylamine and glycol ether DPM solvent, respectively.

Liquid oleylamine (neat) is fed directly to the reactor from a larger oleylamine storage tank in a 1:1 ratio to the MAT28 technical powder and the mixture is stirred with a spin bar agitator. It can be heated up to 40° C. (104° F.) to accelerate the reaction time. The operating range for this stream is 65-130° F. due to the pour point of the oleylamine (59° F.) and the flash point of the glycol ether DPM solvent (167° F.).

The recommended temperature range for the reactor is also 65-130° F. based on the pour point of the unreacted oleylamine (59° F.) and the flash point of the solvent (167° F.). The reaction is completed when no solid particle are present. The finished product is relatively viscous.

### Example 4

### Preparation of the Liquid Intermediate of 6-amino-5-chloro-2-cyclopropylpyrimidine-4-Carboxylic Acid Reacted with Oleylamine (MAT28 Liquid Intermediate) Under Low- or Non-Shear Conditions with Solvent

The 6-amino-5-chloro-2-cyclopropylpyrimidine-4-carboxylic acid (MAT28) active ingredient is supplied as a fine technical powder which is 89.3% pure. In this embodiment, the active ingredient is applied to current base fertilizers by producing a 13% liquid intermediate which would be sprayed onto the base in the blender. The MAT28 liquid intermediate is formed via a weak acid-weak base neutralization reaction between the carboxylic acid group of the MAT28 molecule and the amine functional group of an oleylamine. This reaction forms a salt which, when dissolved in a glycol ether solvent, creates the desired liquid MUP. In the present embodiment of the invention the MAT28 MUP uses a low-shear or a non-shear mixing method.

### Manufacture of 13% MUP

The rate of application of MAT28 onto Northern fertilizer base is 0.065% which can be achieved by applying a 13% MAT28 liquid intermediate onto the base at a level of 0.5% liquid loading. The processes for manufacturing a 13% MAT28 liquid intermediate are outlined below.

### Discharge to Reactor

The reaction vessel is charged with the solvent, for example, glycol ether solvent, that is fed directly to the reaction vessel from a storage tank. The operating range for this stream is also 65-130° F. based on the pour point of oleylamine and the flash point of the glycol ether solvent.

In the next step, a corresponding amount of oleyl amine is added and stirred until homogeneous. The operating range for the oleylamine addition stream is 65-130° F. due to the pour point of the oleylamine (59° F.) and the flash point of the glycol ether DPM solvent (167° F.).

In the next step, the solid MAT 28 technical powder is added to the reactor and the mixture is stirred until no solid particles are present. Specifically, a bulk sack of the MAT28 technical powder per batch is fed to a powder hopper. This hopper discharges the technical powder to the reactor. The operating range for the MAT28 stream going into the reactor is 65-130° F. due to the pour point and flash point of the oleylamine and glycol ether DPM solvent, respectively.

The above mixture in the reactor can be heated up to 40° C. (104° F.) in order to accelerate the reaction time. The mixture is stirred with a spin bar agitator and is adequately mixed. The recommended temperature range for the reactor is also 65-130° F. based on the pour point of the unreacted oleylamine (59° F.) and the flash point of the solvent (167° F.). The reaction is completed when no solid particle are present.

### Formulation/Utility

Compounds of this invention will generally be used as a formulation or composition with an agriculturally suitable carrier comprising at least one of a liquid diluent, a solid diluent or a surfactant. The formulation or composition ingredients are selected to be consistent with the physical properties of the active ingredient, mode of application and environmental factors such as soil type, moisture and temperature. Useful formulations include liquids such as solutions (including emulsifiable concentrates), suspensions, emulsions (including microemulsions and/or suspoemulsions) and the like which optionally can be thickened into gels. Useful formulations further include solids such as dusts, powders, granules, pellets, tablets, films (including seed coatings), and the like which can be water-dispersible (“wettable”) or water-soluble. Active ingredient can be (micro)encapsulated and further formed into a suspension or solid formulation; alternatively the entire formulation of active ingredient can be encapsulated (or “overcoated”). Encapsulation can control or delay release of the active ingredient. Sprayable formulations can be extended in suitable media and used at spray volumes from about one to several hundred liters per hectare. High-strength compositions are primarily used as intermediates for further formulation.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders. | | | |
| Suspensions, Emulsions, Solu- | 1-50 | 40-99 | 0-50 |
| tions (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Typical solid diluents are described in Watkins, et al., Handbook of Insecticide Dust Diluents and Carriers, 2nd Ed., Dorland Books, Caldwell, N.J. Typical liquid diluents are described in Marsden, Solvents Guide, 2nd Ed., Interscience, New York, 1950. McCutcheon's Detergents and Emulsifiers Annual, Allured Publ. Corp., Ridgewood, N.J., as well as Sisely and Wood, Encyclopedia of Surface Active Agents, Chemical Publ. Co., Inc., New York, 1964, list surfactants and recommended uses. All formulations can contain minor amounts of additives to reduce foam, caking, corrosion, microbiological growth and the like, or thickeners to increase viscosity.

Surfactants include, for example, polyethoxylated alcohols, polyethoxylated alkylphenols, polyethoxylated sorbitan fatty acid esters, dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates, glycerol esters, polyoxyethylene/polyoxypropylene block copolymers, and alkylpolyglycosides where the number of glucose units, referred to as degree of polymerization (D.P.), can range from 1 to 3 and the alkyl units can range from C₆ to C₁₄ (see Pure and Applied Chemistry 72, 1255-1264). Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, glycerine, triacetine, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, acetates such as hexyl acetate, heptyl acetate and octyl acetate, and alcohols such as methanol, cyclohexanol, decanol, benzyl and tetrahydrofurfuryl alcohol.

Useful formulations of this invention may also contain materials well known to those skilled in the art as formulation aids such as antifoams, film formers and dyes. Antifoams can include water dispersible liquids comprising polyorganosiloxanes like Rhodorsil® 416. The film formers can include polyvinyl acetates, polyvinyl acetate copolymers, polyvinylpyrrolidone-vinyl acetate copolymer, polyvinyl alcohols, polyvinyl alcohol copolymers and waxes. Dyes can include water dispersible liquid colorant compositions like Prolzed® Colorant Red. One skilled in the art will appreciate that this is a non-exhaustive list of formulation aids. Suitable examples of formulation aids include those listed herein and those listed in McCutcheon's 2001, Volume 2: Functional Materials published by MC Publishing Company and PCT Publication WO 03/024222.

Solutions, including emulsifiable concentrates, can be prepared by simply mixing the ingredients. Dusts and powders can be prepared by blending and, usually, grinding as in a hammer mill or fluid-energy mill. Suspensions are usually prepared by wet-milling; see, for example, U.S. Pat. No. 3,060,084. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modem Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182;

U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

Compounds of the present invention may be highly active preemergent and/or postemergent herbicides and/or plant growth regulants. Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.0001 to 20 kg/ha with a preferred range of about 0.001 to 5 kg/ha and a more preferred range of about 0.004 to 3 kg/ha. One skilled in the art can easily determine the herbicidally effective amount necessary for the desired level of weed control.

Compounds of this invention can be used alone or in combination with other herbicides, insecticides and fungicides, and other agricultural chemicals such as fertilizers. Mixtures of the compounds of the invention with other herbicides can broaden the spectrum of activity against additional weed species, and suppress the proliferation of any resistant biotypes. A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminopyralid, amitrole, ammonium sulfamate, anilofos, asulam, atrazine, azimsulfuron, beflubutamid, benazolin, benazolin-ethyl, benfluralin, benfuresate, bensulfuron-methyl, bensulide, bentazone, benzobicyclon, benzolenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clefoxydim, clethodim, clodinafop-propargyl, clomazone, clomeprop, clopyralid, clopyralid-olamine, cloransulam-methyl, copper sulfate, CUH-35 (2-methoxyethyl 2-[[[4-chloro-2-fluoro-5-[(1-methyl-2-propynyl)oxy]phenyl](3-fluorobenzoyl)amino]carbonyl]-1-cyclohexene-1-carboxylate), cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, epoprodan, EPTC, esprocarb, ethalfluralin, ethametsulfuron-methyl, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupyrsulfuron-methyl and its sodium salt, flurenol, flurenol-butyl, fluridone, fluorochloridone, fluoroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halosulfuron-methyl, haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yl)methyl]-4H-1,2,4-triazole-4-carboxamide), imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, isoxachlortole, isoxadifen, KUH-021 (N-[2-[(4,6-dimethoxy-2-pyrimidinyl)hydroxymethyl]-6-(methoxymethyl)phenyl]-1,1-difluoromethanesulfonamide), lactofen, lenacil, linuron, maleic hydrazide, MCPA and its salts (e.g., MCPAdimethylammonium, MCPA-potassium and MCPA-sodium), esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxyamid, phenmedipham, picloram, picloram-potassium, picolinafen, pinoxaden, piperofos, pretilachlor, primisulfuron-methyl, prodiamine, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen-ethyl, pyrazogyl, pyrazolate, pyrazolynate, pyrazoxyfen, pyrazosulfuron-ethyl, pyribenzoxim, pyributicarb, pyridate, pyriftalid, pyriminobac-methyl, pyrithiobac, pyrithiobac-sodium, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tepraloxydim, terbacil, terbumeton, terbuthylazine, terbutryn, thenylchlor, thiazopyr, thifensulfuron-methyl, thiobencarb, tiocarbazil, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palnmivora (Butyl.) Butyl. and Puccinia thlaspeos Schub. Combinations of compounds of the invention with other herbicides can result in a greater-than-additive (i.e. synergistic) effect on weeds and/or a lessthan-additive effect (i.e. safening) on crops or other desirable plants. In certain instances, combinations with other herbicides having a similar spectrum of control but a different mode of action will be particularly advantageous for preventing the development of resistant weeds. Herbicidally effective amounts of compounds of the invention as well as herbicidally effective amounts of other herbicides can be easily determined by one skilled in the art through simple experimentation.

Preferred for better control of undesired vegetation (e.g., lower use rate, broader spectrum of weeds controlled, or enhanced crop safety) or for preventing the development of resistant weeds are mixtures of a compound of this invention with a herbicide selected from the group consisting of diuron, hexazinone, terbacil, bromacil, glyphosate (particularly glyphosate-isopropylammonium, glyphosate-sodium, glyphosate-potassium, glyphosatetrimesium), glufosinate (particularly glufosinate-ammonium), azimsulfuron, chlorsulfuron, ethametsulfuron-methyl, chlorimuron-ethyl, bensulfuron-methyl, rimsulfuron, sulfometuron-methyl, metsulfuron-methyl, nicosulfuron, tribenuron-methyl, thifensulfuron-methyl, flupyrsulfuron-methyl, flupyrsulfuron-methyl-sodium, halosulfuron-methyl, primisulfuron-methyl, trifloxysulfuron, foramsulfuron, mesosulfuron-methyl, iodosulfuron-methyl, isoproturon, ametryn, amitrole, paraquat dichloride, diquat dibromide, atrazine, metribuzin, acetochlor, metolachlor, S-metolachlor, alachlor, pretilachlor, sethoxydim, tralkoxydim, clethodim, cyhalofop-butyl, quizalofop-ethyl, diclofop-methyl, clodinafop-propargyl, fenoxaprop-ethyl, dimethenamid, flufenacet, picloram, prodiamine, fosamine-ammonium, 2,4-D, 2,4-DB, dicamba, penoxsulam, flumetsulam, naptalam, pendimethalin, oryzalin, MCPA (and its dimethylammonium, potassium and sodium salts), MCPA-isoctyl, MCPA-thioethyl mecoprop, clopyralid, aminopyralid, triclopyr, fluoroxypyr, diflufenzopyr, imazapyr, imazethapyr, imazamox, picolinafen, oxyfluorfen, oxadiazon, carfentrazone-ethyl, sulfentrazone, flumioxazin, diflufenican, bromoxynil, propanil, thiobencarb, molinate, fluridone, mesotrione, sulcotrione, isoxaflutole, isoxaben, clomazone, anilofos, beflubutamid, benfuresate, bentazone, benzobicyclon, benzolenap, bromobutide, butachlor, butamifos, cafenstrole, clomeprop, dimepiperate, dimethametryn, daimuron, esprocarb, etobenzanide, fentrazamid, indanofan, cumylron, menfenacet, oxaziclomefone, oxadiargyl, pentoxazone, pyraclonil, pyrazolate, pyributicarb, pyriftalid, pyriminobac-methyl, thenylchlor, bispyribac-sodium, clefoxydim, copper sulfate, cinosulfuron, cyclosulfamuron, ethoxysulfuron, epoprodan, flucetosulfuron, imazosulfuron, metamifop, pyrazosulfuron-ethyl, quinclorac, flucarbazone-sodium, propoxycarbazone-sodium, amicarbazone, florasulam, triasulfuron, triaziflam, pinoxaden, tritosulfuron, amidosulfuron, metosulam, sulfosulfuron, pyraflufen-ethyl, HOK-201, KUH-021 and CUH-35.

The proportions of the compounds of the invention with other herbicidal active ingredients in herbicidal compositions are generally in the ratio of 100:1 to 1:100, more commonly 10:1 to 1:10 and most commonly 5:1 to 1:5 by weight. The optimum ratios can be easily determined by those skilled in the art based on the weed control spectrum desired.

Particularly noteworthy because of greater than additive (i.e. synergistic) efficacy on certain weeds are mixtures of compounds of the invention with auxin transport inhibitors (phytotropins), an example being the combination of compound 1 (ethyl 6-amino-5-bromo-2-cyclopropyl-4-pyrimidinecarboxylate) with diflufenzopyr. Auxin transport inhibitors are chemical substances that inhibit auxin transport in plants, such as by binding with an auxincarrier protein. Other examples of auxin transport inhibitors include naptalam (also known as N-(1-naphthyl)phthalamic acid and 2-[(1-naphthalenylamino)carbonyl]benzoic acid), 9-hydroxyfluorene-9-carboxylic acid and 2,3,5-triiodobenzoic acid. Therefore an aspect of the present invention relates to a herbicidal mixture comprising synergistically effective amounts of a compound of Claim 1 and an auxin transport inhibitor. Synergistically effective amounts of auxin transport inhibitors with the compounds of the invention can be easily determined.

Compounds of this invention can also be used in combination with herbicide safeners such as benoxacor, BCS (1-bromo-4-[(chloromethyl)sulfonyl]benzene), cloquintocet-mexyl, cyometrinil, dichlormid, 2-(dichloromethyl)-2-methyl-1,3-dioxolane (MG 191), fenchlorazole-ethyl, fenclorim, flurazote, fluxofenim, furilazole, isoxadifen-ethyl, mefenpyrethyl, methoxyphenone ((4-methoxy-3-methylphenyl)(3-methylphenyl)methanone), naphthalic anhydride (1,8-naphthalic anhydride) and oxabetrinil to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

### Biological Examples of the Invention

### Test A

Seeds of barnyardgrass (Echinochloa crus-galli), crabgrass (Digitaria sanguinalis), giant foxtail (Setaria faberi), morningglory (Ipomoea spp.), redroot pigweed (Amnaranthus retroflexus) and velvetleaf (Abutilon theophrasti) are planted into a blend of loam soil and sand and treated preemergence with a directed soil spray using test chemicals formulated in a non-phytotoxic solvent mixture which included a surfactant. At the same time these species are also treated with postemergence applications of test chemicals formulated in the same manner.

Plants range in height from 2 to 10 cm and are in the 1- to 2-leaf stage for the postemergence treatment. Treated plants and untreated controls are maintained in a greenhouse for approximately ten days, after which time all treated plants are compared to untreated controls and visually evaluated for injury.

### Test B

Seeds selected from barnyardgrass (Echinochloa crus-galli), Surinam grass (Brachiaria decumbens), cocklebur (Xanthium strumarium), corn (Zea mays), crabgrass (Digitaria sanguinalis), giant foxtail (Setaria faberii), lambsquarters (Chenopodium album), morningglory (Ipomoea coccinea), pigweed (Amaranthus retroflexus), velvetleaf (Abutilon theophrasti), and wheat (Triticum aestivum) are planted and treated preemergence with test chemicals formulated in a non-phytotoxic solvent mixture which include a surfactant.

At the same time, plants selected from these crop and weed species and also blackgrass (Alopecurus myosuroides) and wild oat (Avenafatua) are treated with postemergence applications of test chemicals formulated in the same manner. Plants range in height from 2 to 18 cm (1- to 4-leaf stage) for postemergence treatments. Plant species in the flooded paddy test consist of rice (Oryza sativa), umbrella sedge (Cyperus diformis), duck salad (Heteranthera limosa) and barnyardgrass (Echinochloa crus-galli) grown to the 2-leaf stage for testing. Treated plants and controls are maintained in a greenhouse for 13 to 15 days, after which time all species are compared to controls and visually evaluated.

### Test C

Seeds or nutlets of plant species selected from bermudagrass (Cynodon dactylon), Surinam grass (Brachiaria decumbens), cocklebur (Xanthium strumarium), corn (Zea mays), crabgrass (Digitaria sanguinalis), woolly cupgrass (Eriochloa villosa), giant foxtail (Setaria faberii), goosegrass (Eleusine indica), johnsongrass (Sorghum halepense), kochia (Kochia scoparia), lambsquarters (Chenopodium album), morningglory (Ipomoea coccinea), eastern black nightshade (Solanum ptycanthum), yellow nutsedge (Cyperus esculentus), pigweed (Amaranthus retroflexus), common ragweed (Ambrosia elatior), soybean (Glycine max), common (oilseed) sunflower (Helianthus annuus), and velvetleaf (Abutilon theophrasti) are planted and treated preemergence with test chemicals formulated in a non-phytotoxic solvent mixture which included a surfactant.

At the same time, plants selected from these crop and weed species and also winter barley (Hordeum vulgare), blackgrass (Alopecurus myosuroides), canarygrass (Phalaris minor), chickweed (Stellaria media), downy brome (Bromus tectorum), green foxtail (Setaria viridis), Italian ryegrass (Lolium multiflorum), wheat (Triticum aestivum), wild oat (Avena fatua) and windgrass (Apera spica-venti) are treated with postemergence applications of some of the test chemicals formulated in the same manner. Plants range in height from 2 to 18 cm (1- to 4-leaf stage) for postemergence treatments. Plant species in the flooded paddy test consist of rice (Oryza sativa), umbrella sedge (Cyperus difformis), duck salad (Heteranthera limosa) and bamyardgrass (Echinochloa crus-galli) grown to the 2-leaf stage for testing. Treated plants and controls are maintained in a greenhouse for 12 to 14 days, after which time all species are compared to controls and visually evaluated.

### Test D

Seeds of plant species selected from catchweed bedstraw (galium; Galium aparine), common chickweed (Stellaria media), kochia (Kochia scoparia), lambsquarters (Chenopodium album), pigweed (Amaranthus retroflexus), Russian thistle (Salsola kali), wild buckwheat (Polygonum convolvulus), wild mustard (Sinapis arvensis), winter barley (Hordeum vulgare), and wheat (Triticum aestivum) are planted and treated preemergence with test chemicals formulated in a non-phytotoxic solvent mixture which includes a surfactant.

At the same time, plants selected from these crop and weed species are treated with postemergence applications of some of the test chemicals formulated in the same manner. Plants range in height from 2 to 18 cm (1- to 4-leaf stage) for postemergence treatments. Treated plants and controls are maintained in a controlled growth environment for 15 to 25 days after which time all species are compared to controls and visually evaluated.

test E

Three plastic pots (ca. 16-cm diameter) per rate are partially filled with sterilized Tama silt loam soil comprising a 35:50:15 ratio of sand, silt and clay and 2.6% organic matter. Separate plantings for each of the three pots are as follows. Seeds from the U.S. of ducksalad (Heteranthera limosa), smallflower umbrella sedge (Cyperus difformis) and purple redstem (Ammannia coccinea), are planted into one 16-cm pot for each rate. Seeds from the U.S. of rice flatsedge (Cyperus iria), bearded (brdd.) sprangletop (Leptochloafusca ssp. fascicularis), one stand of 9 or 10 water seeded rice seedlings (Oryza sativa cv. ‘Japonica M202’), and one stand of 6 transplanted rice seedlings (Oryza sativa cv. ‘Japonica—M202’) are planted into one 16-cm pot for each rate. Seeds from the U.S. of barnyardgrass (Echinochloa crus-galli), late watergrass (Echinochloa oryzicola), early watergrass (Echinochloa oryzoides) and junglerice (Echinochloa colona) are planted into one 16-cm pot for each rate. Plantings are sequential so that crop and weed species are at the 2.0 to 2.5-leaf stage at time of treatment.

Potted plants are grown in a greenhouse with day/night temperature settings of 29.5/26.7° C., and supplemental balanced lighting is provided to maintain a 16-hour photoperiod. Test pots are maintained in the greenhouse until test completion.

At time of treatment, test pots are flooded to 3 cm above the soil surface, treated by application of test compounds directly to the paddy water, and then maintained at that water depth for the duration of the test. Effects of treatments on rice and weeds are visually evaluated by comparison to untreated controls after 21 days.

test F

Seeds or nutlets of plant species selected from (turf) bermudagrass (Cynodon dactylon), Kentucky bluegrass (Poa pratensis), bentgrass (Agrostis palustris), hard fescue (Festuca ovina), large crabgrass (Digitaria sanguinalis), goosegrass (Eleusine indica), dallisgrass (Paspalum dilatatum), annual bluegrass (Poa annua), common chickweed (Stellaria media), dandelion (Taraxacum officinale), white clover (Trifolium repens), and yellow nutsedge (Cyperus esculentus) are planted and treated preemergence with the test chemical formulated in a non-phytotoxic solvent mixture which includes a surfactant.

At the same time, plants selected from these crop and weed species are treated with postemergence applications of the test chemical formulated in the same manner. Plants range in height from 2 to 18 cm (1- to 4-leaf stage) for postemergence treatments. Treated plants and controls are maintained in a greenhouse for 12 to 14 days, after which time all species are compared to controls and visually evaluated.
test G

Seeds or nutlets of plant species selected from bermudagrass (Cynodon dactylon), Surinam grass (Brachiaria decumbens), large crabgrass (Digitaria sanguinalis), green foxtail (Setaria viridis), goosegrass (Eleusine indica), johnsongrass (Sorghum halepense), kochia (Kochia scoparia), pitted momingglory (Ipomoea lacunosa), purple nutsedge (Cyperus rotundus), common ragweed (Ambrosia elatior), black mustard (Brassica nigra), guineagrass (Panicum maximum), dallisgrass (Paspalum dilatatum), barnyardgrass (Echinochloa crus-galli), southern sandbur (Cenchrus echinatus), common sowthistle (Sonchus oleraceous), prickly sida (Sida spinosa), Italian ryegrass (Lolium multiflorum), common purslane (Portulaca oleracea), broadleaf signalgrass (Brachiaria platyphylla), common groundsel (Senecio vulgaris), common chickweed (Stellaria media), tropical spiderwort (Commelina benghalensis), annual bluegrass (Poa annua), downy bromegrass (Bromus tectorum), itchgrass (Rottboellia cochinchinensis), quackgrass (Elytrigia repens), Canada horseweed (Conyza canadensis), field bindweed (Convolvulus arvensis), spanishneedles (Bidens bipinnata), common mallow (Malva sylvestris), and Russian thistle (Salsola kali) are planted and treated preemergence with test chemicals formulated in a non-phytotoxic solvent mixture which includes a surfactant.

At the same time, plants selected from these weed species are treated with postemergence applications of some of the test chemicals formulated in the same manner. Plants range in height from 2 to 18 cm (1- to 4-leaf stage) for postemergence treatments. Treated plants and controls sre maintained in a greenhouse for 12 to 21 days, after which time all species are compared to controls and visually evaluated.

At a different time, established container-grown grape (Vitus vinifera) vines, and olive (Olea europaea) and orange (Citrus sinensis) trees are treated with some of the test chemicals formulated in the same manner and applied to the soil surface and the lower 5 cm of the plant vines or trunks (post-directed application). Plants range in height from 30 to 100 cm. The applications are made using a hand sprayer delivering a volume of 990 L/ha. Treated plants and controls are maintained in a greenhouse for 28 days, after which time the treated plants are compared to controls and visually evaluated.

Also at a different time, seed pieces (nodes) of sugarcane (Saccharum officinarum) are planted and treated preemergence and/or postemergence with some of the test chemicals formulated in the same manner. Treated plants and controls are maintained in a greenhouse for 14 days, after which time the treated plants are compared to controls and visually evaluated.

### Test H

This test evaluates the effect of mixtures of long-chain fatty amine salts of compound in Formula II with diflufenzopyr on several plant species. Seeds of test plants consisting of large crabgrass (DIGSA, Digitaria sanguinalis (L.) Scop.), lambsquarters (CHEAL, Chenopodium album L.), redroot pigweed (AMARE, Amaranthus retroflexus L.), cocklebur (XANST, Xanthium strumarium L.), barnyardgrass (ECHCG; Echinochloa crus-galli (L.) Beauv.), corn (ZEAMD, Zea mays L. cv. ‘Pioneer 33G26’), scarlet (red) morningglory (IPOCO, Ipomoea coccinea L.), giant foxtail (SETFA, Setaria faberi Herrm.) and velvetleaf (ABUTH, Abutilon theophrasti Medik.) are planted in pots containing Redi-Earth® planting medium (Scotts Company, 14111 Scottslawn Road, Marysville, Ohio 43041) comprising spaghnum peat moss, vermiculite, wetting agent and starter nutrients. Seeds of small-seeded species are planted about 1 cm deep; larger seeds were planted about 2.5 cm deep. Plants are grown in a greenhouse using supplemental lighting to maintain a photoperiod of about 14 hours; daytime and nighttime temperatures are about 25-30° C. and 22-25° C., respectively. Balanced fertilizer is applied through the watering system. The plants are grown for 7 to 11 days so that at time of treatment the plants ranges in height from 2 to 18 cm (1- to 4-leaf stage). Treatments consist of fatty amine salts of Compound 1 and diflufenzopyr alone and in combination, suspended or dissolved in an aqueous solvent comprising glycerin and Tween nonionic surfactant and applied as a foliage spray using a volume of 541 L/ha. Each treatment is replicated four times. The application solvent is observed to have no effect compared to untreated check plants. Treated plants and controls are maintained in the greenhouse and watered as needed with care to not wet the foliage for the first 24 hours after treatment. The effects on the plants approximately 3 weeks after treatment are visually compared to untreated controls. Plant response ratings are calculated as the means of the four replicates, based on a scale of 0 to 100 where 0 is no effect and 100 is complete control. Colby's Equation is used to determine the herbicidal effects expected from the mixtures. Colby's Equation (Colby, S. R. “Calculating Synergistic and Antagonistic Responses of Herbicide Combinations,” Weeds, 15(1), pp 20-22 (1967)) calculates the expected additive effect of herbicidal mixtures, and for two active ingredients is of the form:

P_{a+b}=Pₐ+P_{b}−(PₐP_{b}/100)

wherein P_{a+b} is the percentage effect of the mixture expected from additive
contribution of the individual components,
Pₐ is the observed percentage effect of the first active ingredient at the same use rate as in the mixture, and
P_{b} is the observed percentage effect of the second active ingredient at the same use rate as in the mixture.

### Test I

This test evaluates the effect of mixtures of MAT28 liquid intermediate (6-amino-5-chloro-2-cyclopropylpyrimidine-4-carboxylic acid reacted with oleylamine) with metsulfuron-methyl and with a 5:1 by weight combination of chlorsulfuron and metsulfuron-methyl on several plant species. Seeds of test plants consisting of wheat (TRZAW; Triticum aestivum), wild buckwheat, (POLCO; Polygonum convolvulus), redroot pigweed (AMARE; Amaranthus retroflexus), wild mustard (SINAR; Sinapis arvensis), catchweed bedstraw (GALAP; Galiurn aparine), Russian thistle (SASKR; Salsola kali), common chickweed (STEME; Stellaria media), kochia (KCHSC; Kochia scoparia), and lambsquarters (CHEAL; Chenopodium album) are planted into a blend of loam soil and sand. Plants are grown in a greenhouse using supplemental lighting to maintain a photoperiod of about 14 hours; daytime and nighttime temperatures are about 23° C. and 16° C., respectively. Balanced fertilizer is applied through the watering system. The plants are grown for 10 to 23 days so that at time of treatment the plants range from 2- to 8-leaf stage. Treatments consist of MAT28 liquid intermediate, metsulfuron-methyl, and chlorsulfuron-metsulfuron-methyl (5:1) alone and in combination. The treatments are formulated in a non-phytotoxic solvent mixture which includes a surfactant and applied as a foliage spray using a volume of 280-458 L/ha. Each treatment is replicated three times. The application solvent is observed to have no effect compared to untreated check plants. Treated plants and controls are maintained in the greenhouse and watered as needed with care to not wet the foliage for the first 24 hours after treatment. The effects on the plants approximately 17 days after treatment is visually compared to untreated controls. Plant response ratings are calculated as the means of the three replicates, based on a scale of 0 to 100 where 0 is no effect and 100 is complete control. Colby's Equation is used to determine the herbicidal effects expected from the mixtures.

### Test J

This test evaluates the effect of mixtures of MAT28 liquid intermediate (6-amino-5-chloro-2-cyclopropylpyrimidine-4-carboxylic acid reacted with oleylamine)with azimsulfuron on several plant species. Three plastic pots (ca. 16-cm diameter) per rate are partially filled with sterilized Tama silt loam soil comprising a 35:50:15 ratio of sand, silt and clay and 2.6% organic matter. Separate plantings for each of the three pots are as follows. Seeds from the U.S. of ducksalad (HETLI; Heteranthera linosa), smallflower umbrella sedge (CYPDI; Cyperus difformis) and purple redstem (AMMCO; Ammannia coccinea), are planted into one 16-cm pot for each rate. Seeds from the U.S. of bearded sprangletop (LEFUF; Leptochloafusca ssp. fascicularis), one stand of 9 or 10 water-seeded rice seedlings (ORYSW; Oryza sativa cv. ‘Japonica—M202’), and one stand of 6 trans-planted rice seedlings (ORY SP; Oryza sativa cv. ‘Japonica M202’) are planted into one 16-cm pot for each rate. Seeds from the U.S. of barnyardgrass (ECHCG; Echinochloa crus-galli), late watergrass (ECOR2; Echinochloa oryzicola), early watergrass (ECHOR; Echinochloa oryzoides) and junglerice (ECHCO; Echinochloa colona) are planted into one 16-cm pot for each rate. Plantings are sequential so that crop and weed species are at the 2.0 to 2.5-leaf stage at time of treatment.

Potted plants are grown in a greenhouse with day/night temperature settings of 29.5/26.7° C., and supplemental balanced lighting is provided to maintain a 16-hour photoperiod. Test pots are maintained in the greenhouse until test completion.

At time of treatment, test pots are flooded to 3 cm above the soil surface and then treated by application directly to the paddy water of test compounds formulated in a non-phytotoxic solvent mixture which include a surfactant. The pots are maintained at the 3-cm water depth for the duration of the test. Treatments consist of compound 58 and azimsulfuron alone and in combination. Effects of treatments on rice and weeds are visually evaluated by comparison to untreated controls after 21 days. Plant response ratings were calculated as the means of the three replicates.

### Test K

Seeds of plant species selected from sulfonylurea herbicide-susceptible (SUsusceptible) and sulfonylurea herbicide-resistant (SU-resistant) catchweed bedstraw (GALAP; Galium aparine) and wheat (TRZAW; Triticum aestivum) are treated with postemergence applications of test chemicals formulated in a non-phytotoxic solvent mixture 20 which include a surfactant. Plants are treated at the 2-3 leaf stage and 2 whorl stage for wheat and catchweed bedstraw, respectively. Treated plants and controls are maintained in a controlled growth environment for 15 days after which time all species are compared to controls and visually evaluated.

### Test L

This field study includes treatments that consists of MAT28 liquid intermediate (6-amino-5-chloro-2-cyclopropylpyrimidine-4-carboxylic acid reacted with oleylamine)and nicosulfuron alone and in combination on Canada thistle (Cirsium arvense) and daisy fleabane (Erigeron spp.). The plants ranges from 20 to 30 cm in height at the time of application. MAT28 liquid intermediate is formulated as a wettable powder containing 25% active ingredient by weight. Nicosulfuron is in the form of Accent® Herbicide, a water-dispersible granule formulation containing 75% active ingredient by weight. The formulations are dispersed in water in the sprayer tank before treatment. The treatments are made using a backpack sprayer calibrated to deliver 24 gallons per acre (224 L per hectare) to a 10 ft×30 ft (3 m×9 m) plot. Each treatment is replicated two times. The effects on the plants approximately 56 days after treatment are visually compared to untreated controls. Plant response ratings are calculated as the means of the two replicates, based on a scale of 0 to 100 where 0 is no effect and 100 is complete control.

What is claimed is:

1. A process for preparing an intermediate compound, comprising the following steps:
(a) contacting an acid-form of at least one active ingredient compound described in Formula I with at least one long-chain fatty amine, to form an agriculturally suitable salt; and
(b) solubilizing said agriculturally suitable salt of step (a) in at least one solubilizing solvent to form said intermediate compound;
wherein said fatty amine comprises one or more long-chain hydrocarbyl groups having the formula R^{j}R^{k}R^{l}N, in which R^{j}, R^{k}, or R^{l} is H or at least one of R^{j}, R^{k}, or R^{l} is a C₁₂-C₂₂, linear or branched, saturated or unsaturated, substituted or unsubstituted, ethoxylated or non-ethoxylated, propoxylated or non-propoxylated, hydrocarbyl;
wherein said at least one active ingredient is described in Formula I: 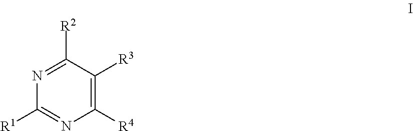
wherein:
R¹ is cyclopropyl optionally substituted with 1-5 R⁵, isopropyl optionally substituted with 1-5 R⁶, or phenyl optionally substituted with 1-3 R⁷;
R² is ((O)ⱼC(R¹⁵)(R¹⁶))ₖR;
wherein R is CO₂H or a herbicidally effective derivative of CO₂H;
R³ is halogen, cyano, nitro, OR²⁰, SR²¹ or N(R²²)R²³;
R⁴ is —N(R²⁴)R²⁵ or —NO₂;
each R⁵ and R⁶ is independently halogen, C₁-C₆ alkyl, C₁-C₆ haloalkyl, C₂-C₆ alkenyl, C₂-C₆ haloalkenyl, C₁-C₃ alkoxy, C₁-C₂ haloalkoxy, C₁-C₃ alkylthio or C₁₋₂ haloalkylthio;
each R⁷ is independently halogen, cyano, nitro, C₁-C₄ alkyl, C₁-C₄ haloalkyl, C₃-C₆ cycloalkyl, C₃-C₆ halocycloalkyl, C₁-C₄ hydroxyalkyl, C₂-C₄ alkoxyalkyl, C₂-C₄ haloalkoxyalkyl, C₂-C₄ alkenyl, C₂-C₄ haloalkenyl, C₃-C₄ alkynyl, C₃-C₄ haloalkynyl, hydroxy, C₁-C₄ alkoxy, C₁-C₄ haloalkoxy, C₂-C₄ alkenyloxy, C₂-C₄ haloalkenyloxy, C₃-C₄ alkynyloxy, C₃-C₄ haloalkynyloxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂-C₄ alkenylthio, C₂-C₄ haloalkenylthio, C₂-C₄ alkenylsulfinyl, C₂-C₄ haloalkenylsulfinyl, C₂-C₄ alkenylsulfonyl, C₂-C₄ haloalkenylsulfonyl, C₃-C₄ alkynylthio, C₃-C₄ haloalkynylthio, C₃-C₄ alkynylsulfinyl, C₃-C₄ haloalkynylsulfinyl, C₃-C₄ alkynylsulfonyl, C₃-C₄ haloalkynylsulfonyl, C₁-C₄ alkylamino, C₂-C₈ dialkylamino, C₃-C₆ cycloalkylamino, C₄-C₆ (alkyl)cycloalkylamino, C₂-C₆ alkylcarbonyl, C₂-C₆ alkoxycarbonyl, C₂-C₆ alkylaminocarbonyl, C₃-C₈ dialkylaminocarbonyl, C₃-C₆ trialkylsilyl, phenyl, phenoxy and 5- or 6-membered heteroaromatic rings, each phenyl, phenoxy and 5- or 6-membered heteroaromatic ring optionally substituted with one to three substituents independently selected from R⁴⁵; or
two adjacent R⁷ are taken together as —OCH₂O—, —CH₂CH₂O—, —OCH(CH₃)O—, —OC(CH₃)₂O—, —OCF₂O—, —CF₂CF₂O—, —OCF₂CF₂O— or —CH═CH—CH═CH—;
R¹⁶ is H, halogen, C₁-C₄ alkyl, C₁-C₄ haloalkyl, hydroxy, C₁-C₄ alkoxy or C₂-C₄ alkylcarbonyloxy;
R¹⁶ is H, halogen, C₁-C₄ alkyl or C₁-C₄ haloalkyl; or
R¹⁵ and R¹⁶ are taken together as an oxygen atom to form, with the carbon atom to which they are attached, a carbonyl moiety;
R²⁰ is H, C₁-C₄ alkyl or C₁-C₃ haloalkyl;
R²¹ is H, C₁-C₄ alkyl or C₁-C₃ haloalkyl;
R²² and R²³ are independently H or C₁-C₄ alkyl;
R²⁴ is H, C₁-C₄ alkyl optionally substituted with 1-2 R³⁰, C₂-C₄ alkenyl optionally substituted with 1-2 R³¹, or C₂-C₄ alkynyl optionally substituted with 1-2 R³²; or R²⁴ is C(═O)R³³, nitro, OR³⁴, S(O)₂R³⁵, N(R³⁶)R³⁷ or N═C(R⁶²)R⁶³;
R²⁵ is H, C₁-C₄ alkyl optionally substituted with 1-2 R³⁰ or C(═O)R³³; or
R²⁴ and R²⁵ are taken together as a radical selected from —(CH₂)₄—, —(CH₂)₅—, —CH₂CH═CHCH₂— and —(CH₂)₂O(CH₂)₂—, each radical optionally substituted with 1-2 R³⁸; or
R²⁴ and R²⁵ are taken together as ═C(R³⁹)N(R⁴⁰)R⁴¹ or ═C(R⁴²)OR⁴³;
each R³⁰, R³¹ and R³² is independently halogen, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
each R³³ is independently H, C₁-C₁₄ alkyl, C₁-C₃ haloalkyl, C₁-C₄ alkoxy, phenyl, phenoxy or benzyloxy;
R³⁴ is H, C₁-C₄ alkyl, C₁-C₃ haloalkyl or CHR⁶⁶C(O)OR⁶⁷;
R³⁵ is C₁-C₄ alkyl or C₁-C₃ haloalkyl;
R³⁶ is H, C₁-C₄ alkyl or C(═O)R⁶⁴;
R³⁷ is H or C₁-C₄ alkyl;
each R³⁸ is independently halogen, C₁-C₃ alkyl, C₁-C₃ alkoxy, C₁-C₃ haloalkoxy, C₁-C₃ alkylthio, C₁-C₃ haloalkylthio, amino, C₁-C₃ alkylamino, C₂-C₄ dialkylamino or C₂-C₄ alkoxycarbonyl;
R³⁹ is H or C₁-C₄ alkyl;
R⁴⁰ and R⁴¹ are independently H or C₁-C₄ alkyl; or
R⁴⁰ and R⁴¹ are taken together as —(CH₂)₄—, —(CH₂)₅—, —CH₂CH═CHCH₂— or —(CH₂)₂O(CH₂)₂—;
R⁴² is H or C₁-C₄ alkyl;
R⁴³ is C₁-C₄ alkyl;
each R⁴⁵ is independently halogen, cyano, nitro, C₁-C₄ alkyl, C₁-C₄ haloalkyl, C₃-C₆ cycloalkyl, C₃-C₆ halocycloalkyl, C₂-C₄ alkenyl, C₂-C₄ haloalkenyl, C₃-C₄ alkynyl, C₃-C₄ haloalkynyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ alkylamino, C₂-C₈ dialkylamino, C₃-C₆ cycloalkylamino, C₄-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁶² is H, C₁-C₄ alkyl or phenyl optionally substituted with 1-3 R⁶⁵;
R⁶³ is H or CIC4 alkyl; or
R⁶² and R⁶³ are taken together as —(CH₂)₄— or —(CH₂)₅—;
R⁶⁴ is H, C₁-C₁₄ alkyl, C₁-C₃ haloalkyl, C₁-C₄ alkoxy, phenyl, phenoxy or benzyloxy;
each R⁶⁵ is independently CH₃, Cl or OCH₃;
R⁶⁶ is H, C₁-C₄ alkyl or C₁-C₄ alkoxy;
R⁶⁷ is H, C₁-C₄ alkyl or benzyl;
j is 0 or 1; and
k is 0 or 1;
provided that:
(a) when k is 0, thenj is 0;
(b) when R² is CH₂OR^{a} wherein R^{a} is H, optionally substituted alkyl or benzyl, then R³ is other than cyano;
(c) when R¹ is phenyl substituted by Cl in each of the meta positions, the phenyl is also substituted by R⁷ in the para position;
(d) when R¹ is phenyl substituted by R⁷ in the para position, said R⁷ is other than tert-butyl, cyano or optionally substituted phenyl;
(e) when R¹ is cyclopropyl or isopropyl optionally substituted with 1-5 R⁶, then R is other than C(═W)N(R^{b})S(O)₂—R^{c}-R^{d} wherein W is O, S, NRe or NOR^{e}; R^{b} is hydrogen, C₁-C₄ alkyl, C₂-C₆ alkenyl or C₂-C₆ alkynyl; R^{c} is a direct bond or CHR^{f}, O, NR^{e} or NOR^{e}; R^{d} is an optionally substituted heterocyclic or carbocyclic aromatic radical having 5 to 6 ring atoms, the radical being optionally condensed with an aromatic or nonaromatic 5- or 6-membered ring; each R^{e} is independently H, C₁-C₃ alkyl, C₁-C₃ haloalkyl or phenyl; and R^{f} is H, C₁-C₃ alkyl or phenyl; and
(f) the compound of Formula I is other than diethyl 6-amino-5-nitro-2-phenyl-4-pyrimidinemalonate.

2. The process as recited in claim 1, wherein said acid-form of at least one active ingredient compound described in Formula I is contacted with said at least one long-chain fatty amine under high shear condition.

3. The process as recited in claim 2, wherein:
R² is CO₂R¹², CH₂OR¹³, CH(OR⁴⁶)(OR⁴⁷), CHO, C(═NOR¹⁴)H, C(═NNR⁴⁸R⁴⁹)H, (O)C(R¹⁵)(R¹⁶)CO₂R¹⁷, C(═O)N(R¹⁸)R¹⁹, C(═S)OR⁵⁰, C(═O)SR⁵¹, C(═S)SR⁵² or C(═NR⁵³)_{y}R⁵⁴;
R¹² is H, —CHC(O)O(CH₂)ₘ, —N═C(R⁵⁵)R⁵⁶; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl, C₂-C₁₄ alkynyl and phenyl, each radical optionally substituted with 1-3 R²⁷; or
R¹² is a divalent radical linking the carboxylic ester function CO₂R¹² of each of two pyrimidine ring systems of Formula I, the divalent radical selected from —CH₂—, —(CH₂)₂—, (CH₂)₃— and —CH(CH₃)CH₂—;
R¹³ is H, C₁-C₁₀ alkyl optionally substituted with 1-3 R²⁸, or benzyl;
R¹⁴ is H, C₁-C₄ alkyl, C₁-C₄ haloalkyl or benzyl;
R¹⁷ is C₁-C₁₀ alkyl optionally substituted with 1-3 R²⁹, or benzyl;
R¹⁸ is H, C₁-C₄ alkyl, hydroxy, Cp-C4 alkoxy or S(O)₂R⁵⁷;
R¹⁹ is H or C₁-C₄ alkyl;
each R²⁷ is independently halogen, cyano, hydroxycarbonyl, C₂-C₄ alkoxycarbonyl, hydroxy, C₁-C₄ alkoxy, C₁-C₄ haloalkoxy, C₁-C₄ alkylthio, C₁-C₄ haloalkylthio, amino, C₁-C₄ alkylamino, C₂-C₄ dialkylamino, —CHO(CH₂)ₙ or phenyl optionally substituted with 1-3 R⁴⁴; or
two R²⁷ are taken together as —OC(O)O— or —O(C(R⁵⁸)(R⁵⁸))₁₋₂O—; or
two R²⁷ are taken together as an oxygen atom to form, with the carbon atom to which they are attached, a carbonyl moiety;
each R²⁸ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino; or
two R²⁸ are taken together as an oxygen atom to form, with the carbon atom to which they are attached, a carbonyl moiety;
each R²⁹ is independently halogen, C₁-C₄ alkoxy, C₁-C₄ haloalkoxy, C₁-C₄ alkylthio, C₁-C₄ haloalkylthio, amino, C₁-C₄ alkylamino or C₂-C₄ dialkylamino;
each R⁴⁴ is independently halogen, C₁-C₄ alkyl, C₁-C₃ haloalkyl, hydroxy, C₁-C₄ alkoxy, C₁-C₃ haloalkoxy, C₁-C₃ alkylthio, C₁-C₃ haloalkylthio, amino, C₁-C₃ alkylamino, C₂-C₄ dialkylamino or nitro;
R⁴⁶ and R⁴⁷ are independently C₁-C₄ alkyl or C₁-C₃ haloalkyl; or
R⁴⁶ and R⁴⁷ are taken together as —CH₂CH₂—, —CH₂CH(CH₃)— or —(CH₂)₃—;
R⁴⁸ is H, C₁-C₄ alkyl, C₁-C₄ haloalkyl, C₂-C₄ alkylcarbonyl, C₂-C₄ alkoxycarbonyl or benzyl;
R⁴⁹ is H, C₁-C₄ alkyl or C₁-C₄ haloalkyl;
R⁵⁰, R⁵¹ and R⁵² are H; or a radical selected from C₁-C₁₄ alkyl, C₃-C₁₂ cycloalkyl, C₄-C₁₂ alkylcycloalkyl, C₄-C₁₂ cycloalkylalkyl, C₂-C₁₄ alkenyl and C₂-C₁₄ alkynyl, each radical optionally substituted with 1-3 R²⁷;
Y is O, S or NR⁶¹;
R⁵³ is H, C₁-C₃ alkyl, C₁-C₃ haloalkyl, C₂-C₄ alkoxyalkyl, OH or C₁-C₃ alkoxy;
R⁵⁴ is C₁-C₃ alkyl, C₁-C₃ haloalkyl or C₂-C₄ alkoxyalkyl; or
R⁵³ and R⁵⁴ are taken together as —(CH₂)₂—, —CH₂CH(CH₃)— or —(CH₂)₃—;
R⁵⁵ and R⁵⁶ are independently C₁-C₄ alkyl;
R⁵⁷ is C₁-C₄ alkyl, C₁-C₃ haloalkyl or NR⁵⁹R⁶⁰;
each R⁵⁸ is independently selected from H and C₁-C₄ alkyl;
R⁵⁹ and R⁶⁰ are independently H or C₁-C₄ alkyl;
R⁶¹ is H, C₁-C₃ alkyl, C₁-C₃ haloalkyl or C₂-C₄ alkoxyalkyl;
m is an integer from 2 to 3; and
n is an integer from 1 to 4.

4. The process as recited in claim 3, wherein R³ is halogen.

5. The process as recited in claim 3, wherein R¹ is cyclopropyl or phenyl substituted with a halogen, methyl or methoxy radical in the para position and optionally with 1-2 radicals selected from halogen and methyl in other positions; and R⁴ is —N(R²⁴)R²⁵.

6. The process as recited in claim 5, wherein R² is CO₂R¹², CH₂OR¹³, CHO or CH₂CO₂R¹⁷.

7. The process as recited in claim 6, wherein R²⁴ is H, C(O)R³³ or C₁-C₄ alkyl optionally substituted with R³⁰; R²⁵ is H or C₁-C₂ alkyl; or R²⁴ and R²⁵ are taken together as ═C(R³⁹)N(R⁴⁰)R⁴¹.

8. The process as recited in claim 7, wherein R² is CO₂R¹²; and R²⁴ and R²⁵ are H.

9. The process as recited in claim 8, wherein R¹² is H, C₁-C₄ alkyl or benzyl.

10. The process as recited in claim 1, wherein said at least one active ingredient compound is selected from the group consisting of:
methyl 6-amino-5-bromo-2-cyclopropyl-4-pyrimidinecarboxylate,
ethyl 6-amino-5-bromo-2-cyclopropyl-4-pyrimidinecarboxylate,
phenylmethyl 6-amino-5-bromo-2-cyclopropyl-4-pyrimidinecarboxylate,
6-amino-5-bromo-2-cyclopropyl-4-pyrimidinecarboxylic acid monosodium salt,
methyl 6-amino-5-chloro-2-cyclopropyl-4-pyrimidinecarboxylate,
phenylmethyl 6-amino-5-chloro-2-cyclopropyl-4-pyrimidinecarboxylate,
6-amino-5-chloro-2-cyclopropyl-4-pyrimidinecarboxylie acid monosodium salt,
ethyl 6-amino-5-chloro-2-cyclopropyl-4-pyrimidinecarboxylate,
methyl 6-amino-5-chloro-2-(4-chlorophenyl)-4-pyrimidinecarboxylate,
ethyl 6-amino-5-chloro-2-(4-chlorophenyl)-4-pyrimidinecarboxylate,
6-amino-5-chloro-2-(4-chlorophenyl)-4-pyrimidinecarboxylic acid,
ethyl 6-amino-2-(4-bromophenyl)-5-chloro-4-pyrimidinecarboxylate,
methyl 6-amino-2-(4-bromophenyl)-5-chloro-4-pyrimidinecarboxylate, and
6-amino-2-(4-bromophenyl)-5-chloro-4-pyrimidinecarboxylic acid.

11. The process recited in claim 1, wherein said at least one active ingredient compound is aminocyclopyrachlor described in Formula I, and said at least one long-chain fatty amine is oleyl amine.

12. A herbicidal mixture comprising a herbicidally effective amount of said intermediate compound prepared by the process of claim 2 and an effective amount of at least one additional active ingredient selected from the group consisting of another herbicide and a herbicide safener.

13. A herbicidal mixture comprising synergistically effective amounts of said intermediate compound prepared by the process of claim 2 and an auxin transport inhibitor.

14. A herbicidal composition comprising a herbicidally effective amount of said intermediate compound prepared by the process of claim 2 and at least one of a surfactant, a solid diluent or a liquid diluent.

15. A method for controlling the growth of undesired vegetation comprising contacting the vegetation or its environment with a herbicidally effective amount of said intermediate compound prepared by the process of claim 2.

16. A method for controlling the growth of undesired vegetation comprising contacting the vegetation or its environment with a herbicidally effective amount of said intermediate compound prepared by the process of claim 2.

17. A herbicidal composition comprising a herbicidally effective amount of said intermediate compound prepared by the process of claim 2, an effective amount of at least one additional active ingredient selected from the group consisting of another herbicide and a herbicide safener, and at least one of a surfactant, a solid diluent or a liquid diluent.

18. The process as recited in claim 17, wherein the additional active ingredient is selected from the group consisting of: amidosulfuron, azimsulfuron, bensulfuron-methyl, bispyribac, bispyribac-sodium, chlorimuron-ethyl, chlorsulfuron, cinosulfuron, cloransulam-methyl, cyclosulfamuron, diclosulam, ethametsulfuron-methyl, ethoxysulfuron, flazasulfuron, florasulam, flucarbazone, flucarbazone-sodium, flucetosulfuron, flumetsulam, flupyrsulfuron-methyl, flupyrsulfuron-methyl-sodium, foramsulfuron, halosulfuron-methyl, imazamethabenz-methyl, imazamox, imazapic, imazapyr, imazaquin, imazaquin-ainmonium, imazethapyr, imazosulfuron, iodosulfuron-methyl, mesosulfuron-methyl, metosulam, metsulfuron-methyl, nicosulfuron, oxasulfuron, penoxsulam, primisulfuron-methyl, propoxycarbazone, propoxycarbazone-sodium, prosulfuron, pyrazosulfuron-ethyl, pyribenzoxim, pyriftalid, pyriminobac-methyl, pyrithiobac, pyrithiobac-sodium, rimsulfuron, sulfometuron-methyl, sulfosulfuron, thifensulfuron-methyl, triasulfuron, tribenuron-methyl, trifloxysulfuron, triflusulfuron-methyl and tritosulfuron.

19. The herbicidal mixture of claim 17 wherein the additional active ingredient is in combination with at least one other active ingredient to form a combination of active ingredients selected from the group consisting of: chlorsulfuron and flucarbazone-sodium; chlorsulfuron and sulfometuron-methyl; flumetsulam, nicosulfuron and rimsulfuron; mesosulfuron-methyl and iodosulfuron-methyl; metsulfuron-methyl and chlorsulfuron; metsulfuron-methyl and sulfometuron-methyl; metsulfuron-methyl, thifensulfuron-methyl and tribenuron-methyl; imazapyr and metsulfuron-methyl; imazapyr, metsulfuron-methyl and sulfometuron-methyl; imazapyr and sulfometuron-methyl; rimsulfuron and nicosulfuron; rimsulfuron and thifensulfuron-methyl; thifensulfuron-methyl and metsulfuron-methyl; tribenuron-methyl and metsulfuron-methyl; tribenuron-methyl and thifensulfuron-methyl; bensulfuron-methyl and metsulfuron-methyl; and metsulfuron-methyl and chlorimuron-ethyl.

20. The herbicidal mixture of claim 13, wherein said active ingredient compound is selected from the group consisting of: ethyl 6-amino-5-chloro-2-cyclopropyl-4-pyrimidinecarboxylate, methyl 6-amino-5-chloro-2-cyclopropyl-4-pyrimidinecarboxylate and 6-amino-5-chloro-2-cyclopropyl-4-pyrimidinecarboxylic acid, and the auxin transport inhibitor is diflufenzopyr.

21. The herbicidal mixture of claim 13, wherein the compound is ethyl 6-amino-5-bromo-2-cyclopropyl-4-pyrimidinecarboxylate and the auxin transport inhibitor is diflufenzopyr.
